(19) United States
(12) Patent Application Publication
(10) Pub. No.: US 2023/0295273 A1
(43) Pub. Date: Sep. 21, 2023

(54) METHODS FOR MODULATING HOST CELL SURFACE INTERACTIONS WITH HUMAN CYTOMEGALOVIRUS

(71) Applicant: Genentech, Inc., South San Francisco, CA (US)

(72) Inventors: Claudio CIFERRI, South San Francisco, CA (US); Marc KSCHONSAK, South San Francisco, CA (US)

(21) Appl. No.: 18/324,406

(22) Filed: May 26, 2023

Related U.S. Application Data
(63) Continuation of application No. PCT/US2021/060887, filed on Nov. 26, 2021.
(60) Provisional application No. 63/118,859, filed on Nov. 27, 2020.

(51) Int. Cl. C07K 16/08; C07K 14/71; C12N 15/113; A61P 31/22

(52) U.S. Cl. CPC C07K 16/085; C07K 14/71; C12N 15/1133; A61P 31/22; C07K 2317/31; C12N 2310/11; C12N 2310/14

(57) ABSTRACT

Provided herein are methods of treating or preventing human cytomegalovirus (HCMV) infection comprising modulating interactions between the HCMV gHgLgO trimer and plasma membrane-expressed host cell proteins, as well as methods of identifying modulators of such interactions.

### CROSS-REFERENCE TO RELATED APPLICATIONS

This application is a continuation of International Patent Application No. PCT/US2021/060887, filed on Nov. 26, 2021, which claims benefit to U.S. Provisional Application No. 63/118,859, filed on Nov. 27, 2020, the entire contents of which are incorporated herein by reference in their entirety.

### SEQUENCE LISTING

The instant application contains a Sequence Listing which has been submitted electronically in XML format and is hereby incorporated by reference in its entirety. Said XML copy, created on May 12, 2023, is named 50474-247003_Sequence_Listing_5_12_23.xml and is 14,931 bytes in size.

### FIELD OF THE INVENTION

Provided herein are methods of treating or preventing human cytomegalovirus (HCMV) infection comprising modulating interactions between the HCMV gHgLgO trimer and plasma membrane-expressed host cell proteins, as well as methods of identifying modulators of such interactions.

### BACKGROUND

Human cytomegalovirus (HCMV) is a member of the Betaherpesvirinae sub-family of Herpesviridae that establishes a life-long infection in more than 70% of the human population. After primary infection, HCMV becomes latent, and its reactivation causes severe morbidity and mortality in individuals who are immune-suppressed or undergoing organ or hematopoietic stem cell (HSC) transplantation. HCMV is particularly threatening during pregnancy due to its ability to cross the placental barrier and infect the fetus. HCMV infection affects 0.3% to 2.3% of newborns, representing the leading viral cause of congenital birth defects, including brain damage, hearing loss, learning disabilities, heart diseases and mental retardation. For these reasons, HCMV has been identified as a top priority disease target by the Institute of Medicine. An effective anti-viral therapeutic or vaccine should target the early steps of the HCMV infection cycle, including viral entry into host cells. HCMV uses several envelope glycoprotein complexes to enter different cell lines, including two gHgL envelope glycoprotein complexes, the gHgLgO (trimer) and the gHgLpUL128-131A (pentamer), as well as glycoprotein B (gB). HCMV trimer or pentamer binding to cellular host receptors provide the triggering signal, through a mechanism yet to be identified, for the HCMV glycoprotein gB to catalyze membrane fusion between the virus and infected cells. This fusion allows HCMV to enter cells, replicate, and establish its latency.

HCMV exhibits a broad cellular tropism, including fibroblasts, monocytes, macrophages, neurons, epithelial and endothelial cells, via interactions with structurally and functionally distinct receptor proteins. Recent evidence suggested a primary role of the trimer complex for the infection of all cell types. The trimer-mediated infection of fibroblasts has been best studied and involves the interaction of the trimer with PDGFRα, a member of the receptor tyrosine kinase 3 (RTK3) family. TGFβR3 was also found to bind the HCMV trimer with high affinity, representing an additional putative cellular receptor that could explain the broad cellular tropism of HCMV.

During the past decades, significant efforts have been established to develop vaccine candidates against HCMV infection. However, results from recent clinical trials indicated that HCMV vaccines showed only modest efficacy in preventing viral infection. Therefore, the development of effective therapeutics against HCMV represents an important unmet medical need.

### SUMMARY OF THE INVENTION

In one aspect, the disclosure features a modulator of the interaction between the gO subunit of the human cytomegalovirus (HCMV) gHgLgO trimer and PDGFRα that binds to the glycosylation-free surface of the gO subunit and causes a decrease in the binding of the gO subunit to PDGFRα.

In some aspects, the modulator binds to (a) one or more of residues R230, R234, V235, K237, and Y238 of the gO subunit; (b) one or more of residues N81, L82, M84, M86, F109, F111, T114, Q115, R117, K121, and V123 of the gO subunit; and (c) one or more of residues R336, Y337, K344, D346, N348, E354, and N358 of the gO subunit.

In another aspect, the disclosure features a modulator of the interaction between the gO subunit of the HCMV gHgLgO trimer and PDGFRα that binds to (a) one or more of residues R230, R234, V235, K237, and Y238 of the gO subunit; (b) N81, L82, M84, M86, F109, F111, T114, Q115, R117, K121, and V123 of the gO subunit; and (c) one or more of residues R336, Y337, K344, D346, N348, E354, and N358 of the gO subunit and causes a decrease in the binding of the gO subunit to PDGFRα.

In some aspects, the modulator binds to all 23 of residues R230, R234, V235, K237, Y238, N81, L82, M84, M86, F109, F111, T114, Q115, R117, K121, V123, R336, Y337, K344, D346, N348, E354, and N358 of the gO subunit.

In some aspects, the modulator further binds to one or more of residues R47, Y84, and N85 of the gH subunit of HCMV.

In some aspects, the modulator is a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody or a multispecific antibody. In some aspects, the bispecific antibody or multispecific antibody binds to at least three distinct epitopes of the gO subunit. In some aspects, the at least three distinct epitopes comprise (a) a first epitope comprising one or more of residues R230, R234, V235, K237, and Y238 of the gO subunit; (b) a second epitope comprising one or more of residues N81, L82, M84, M86, F109, F111, T114, Q115, R117, K121, and V123 of the gO subunit; and (c) a third epitope comprising one or more of residues R336, Y337, K344, D346, N348, E354, and N358 of the gO subunit.

In some aspects, the modulator is a mimic of PDGFRα.

In another aspect, the disclosure features a modulator of the interaction between the gO subunit of the HCMV gHgLgO trimer and PDGFRα that binds to the D1 (SEQ ID NO: 11), D2 (SEQ ID NO: 12), and D3 (SEQ ID NO: 13) domains of PDGFRα and causes a decrease in the binding of the gO subunit to PDGFRα.

In some aspects, the modulator binds to (a) one or more of residues N103, Q106, T107, E108, and E109 of PDGFRα; (b) one or more of residues M133, L137, I139, E141, I147, S145, Y206, and L208 of PDGFRα; and (c) one or more of residues N240, D244, Q246, T259, E263 and K265 of PDGFRα.

In another aspect, the disclosure features a modulator of the interaction between the gO subunit of the HCMV gHgLgO trimer and PDGFRα that binds to (a) one or more of residues N103, Q106, T107, E108, and E109 of PDGFRα; (b) one or more of residues M133, L137, I139, E141, I147, S145, Y206, and L208 of PDGFRα; and (c) one or more of residues N240, D244, Q246, T259, E263 and K265 of PDGFRα and causes a decrease in the binding of the gO subunit to PDGFRα.

In some aspects, the modulator binds to all ten of residues T107, E108, E109, M133, L137, I139, Y206, L208, E263, and K265 of PDGFRα.

In some aspects, the modulator further binds to one or more of residues E52, S78, and L80 of PDGFRα.

In some aspects, the modulator is a small molecule, an antibody or antigen-binding fragment thereof, a peptide, a mimic, or an inhibitory nucleic acid. In some aspects, the inhibitory nucleic acid is an ASO or an siRNA.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody or a multispecific antibody. In some aspects, the bispecific antibody or multispecific antibody binds to at least three distinct epitopes of PDGFRα. In some aspects, the at least three distinct epitopes comprise (a) a first epitope comprising one or more of residues N103, Q106, T107, E108, and E109 of PDGFRα; (b) a second epitope comprising one or more of residues M133, L137, I139, E141, I147, S145, Y206, and L208 of PDGFRα; and (c) a third epitope comprising one or more of residues N240, D244, Q246, T259, E263 and K265 of PDGFRα.

In some aspects, the modulator is a mimic of the gO subunit of the HCMV gHgLgO trimer.

In some aspects, the modulator decreases binding of the gO subunit of the HCMV gHgLgO trimer to PDGFRα by at least 50%. In some aspects, the modulator decreases binding of the gO subunit of HCMV trimer to PDGFRα by at least 90%.

In some aspects, the modulator decreases binding of the gO subunit of the HCMV gHgLgO trimer to TGFβR3 by at least 50%.

In some aspects, the decrease in binding is measured by surface plasmon resonance, biolayer interferometry, or an enzyme-linked immunosorbent assay (ELISA).

In some aspects, the modulator has minimal binding with a region of PDGFRα that triggers downstream signaling.

In some aspects, the modulator does not bind to a region of PDGFRα that triggers downstream signaling.

In some aspects, the region of PDGFRα that triggers downstream signaling is a binding site of PDGF.

In some aspects, the modulator causes less than a 20% decrease in signaling by PDGFRα compared to signaling in the absence of the modulator.

In some aspects, the modulator does not cause a decrease in signaling by PDGFRα compared to signaling in the absence of the modulator.

In some aspects, the modulator causes a decrease in infection of a cell by HCMV relative to infection in the absence of the modulator. In some aspects, infection is decreased by at least 40%, as measured in a viral infection assay or a viral entry assay using pseudotyped particles.

In some aspects, the modulator further comprises a pharmaceutically acceptable carrier.

In another aspect, the disclosure features a method for treating an HCMV infection in an individual, the method comprising administering to the individual an effective amount of a modulator provided herein, thereby treating the individual. In some aspects, the duration or severity of HCMV infection is decreased by at least 40% relative to an individual who has not been administered the modulator.

In another aspect, the disclosure features a method for preventing an HCMV infection in an individual, the method comprising administering to the individual an effective amount of a modulator provided herein, thereby preventing an HCMV infection in the individual.

In another aspect, the disclosure features a method of prophylaxis against a secondary HCMV infection in an individual, the method comprising administering to the individual an effective amount of a modulator provided herein, thereby preventing a secondary HCMV infection in the individual. In some aspects, the secondary infection is an HCMV infection of an uninfected tissue. In some aspects, the individual is immunocompromised, is pregnant, or is an infant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is an overall cryo-electron microscopy (cryo-EM) map showing the human cytomegalovirus (HCMV) gHgLgO glycoprotein trimer complex bound to the neutralizing Fabs 13H111 and Msl-109. Red: gO subunit. Pink: gL subunit. Blue: gH subunit. Gray (left): Fab 13H111. Gray (right): Fab Msl-109.

FIG. 1B is a pair of ribbon diagrams showing the front view (left) and back view (right) of the HCMV gHgLgO trimer complex. Red: gO subunit. Pink: gL subunit. Blue: gH subunit.

FIG. 1C is a pair of diagrams showing the electrostatic surface of the HCMV gHgLgO trimer complex (same views as in FIG. 1B) in the range of −10 to +10 keV. Red: negatively charged. Blue: positively charged.

FIG. 1D is a pair of diagrams showing the distribution of glycosylation sites (colored) of the HCMV gHgLgO trimer complex (same views as in FIG. 1B).

FIG. 2A is a diagram showing the superposition of the gHgL subunits of the HCMV trimer complex (colored) onto the gHgL subunits of the HCMV pentamer complex (gray, PDB code: 5VOB).

FIG. 2B is a diagram showing the superposition of the HCMV trimer gHgL glycosylation sites (colored) onto the HCMV pentamer gHgL glycosylation sites (gray, PDB code: 5VOB).

FIG. 2C is a pair of diagrams showing the front view of HCMV trimer distal region showing the gH N terminus, gL and gO (top panel) and a close-up view of the gL-gO interaction region highlighting the gL loop between residues A131 and V151 and the disulfide bond between gL residue C144 and gO residue C343 (bottom panel). Red: gO subunit. Pink (top panel): gL subunit. Blue: gH subunit.

FIG. 2D is a pair of diagrams showing the front view of the HCMV pentamer distal region showing the gH N-terminus, gL, UL130, UL131 and UL128 (top panel) and a close-up view of the gL-UL128 interaction region highlighting the gL helix between residues A131 and V151 and the disulfide bond between gL residue C144 and UL128 residue C162 (bottom panel). Pink (top panel): gL subunit. Blue: UL131. Green: UL128. Orange: UL130.

FIG. 3A is a diagram showing the front view of HCMV gHgLgO trimer complex bound to the neutralizing Fabs 13H11 and Msl-109. Red: gO subunit. Pink: gL subunit. Blue: gH subunit. Green and light green: 13H11. Orange and light orange: Msl-109.

FIG. 3B is a set of diagrams showing the variable Fab regions of 13H11 and Msl-109 bound to the C-terminal region of gH. Inset panels 1-3 show close-up views of key interaction sites between 13H11 and the HCMV trimer gH subunit. Inset panel 4 shows a close-up view of the key interaction region between Msl-109 and the HCMV trimer gH subunit. Fab contact regions on gH are highlighted in pink. Green: 13H11. Orange: Msl-109.

FIG. 3C is a set of diagrams showing the variable Fab regions for 13H11 and the highlighted interaction surface for the 13H11 heavy (dark green) and light (light green) chains on gH (left), and a close-up view of the variable Fab regions for Msl-109 and the highlighted interaction surface for the Msl-109 heavy (dark orange) and light (light orange) chain on gH (right).

FIG. 4A is a diagram showing the structure of the HCMV gO subunit with domain organization indicated in color.

FIG. 4B is a schematic diagram showing the domain organization of the HCMV gO subunit with secondary structure elements indicated. Domains 1-5: N-terminal beta strands. Domains 6, 9-10, 12: central alpha helices. Domains 16-17: C-terminal alpha helices.

FIG. 4C is a pair of diagrams showing the C-terminal domain of the HCMV gO subunit (left) and the short-chain cytokine fold of the FLT3 ligand (right; PDB code: 3QS7) for structural comparison. Helices shown in pink represent the regions of gO and FLT3 that fold into a cytokine domain.

FIG. 4D is a diagram showing the distribution of cysteine residues (pink) and disulfide bonds within the HCMV gO subunit.

FIG. 4E is a pair of diagrams showing the electrostatic surface of the HCMV gO subunit in the range of −10 to +10 keV. Red: negatively charged. Blue: positively charged.

FIG. 4F is a pair of diagrams showing the results of a conservation analysis of the HCMV gO subunit based on sequences from 93 Herpesvirus 5 strains. Conservation: low to high (green to purple).

FIG. 4G is a pair of diagrams showing the distribution of glycosylation sites (colored) on the HCMV gO subunit.

FIG. 5A is a graph showing the level of HCMV trimer (strains: Merlin and VR1814) binding (normalized binding signal of HCMV trimer, expressed as percent of maximum signal) with the indicated human receptor proteins of the Cell-Surface Receptor Discovery Platform results.

FIG. 5B is a schematic diagram showing the domain organization of human PDGFRα. Domains: D1, D2, D3, D4, D5, transmembrane (TM) domain, and kinase domain.

FIG. 5C is a diagram showing the front view of HCMV gHgLgO trimer complex bound to PDGFRα domains D1-D3. Green: PDGFRα. Red: gO subunit. Pink: gL subunit. Blue: gH subunit.

FIG. 5D is a set of diagrams showing a view of the HCMV trimer distal region including gO, gL and the gH N-terminus and PDGFα D1-D4. PDGFRα D4 is shown with low opacity to illustrate the orientation of the receptor relative to the host-cell membrane. Bottom panels show Site 1-4 residue interactions. Green: D1-D4 of PDGFRα. Red: gO subunit. Pink: gL subunit. Blue: gH subunit.

FIG. 5E is a diagram showing HCMV trimer distal region in a close-up view as described in FIG. 5D, with the highlighted surface area (green) involved in interaction with PDGFRα.

FIG. 5F is a diagram showing the results of a conservation analysis of the HCMV gO-gL+gH N-terminus based on sequences from 93 Herpesvirus 5 strains for gO and gH, and 59 Herpesvirus 5 strains for gL. Conservation range: low to high (green to purple).

FIG. 5G is a bar graph showing the level of binding of HCMV gHgLgO trimer to PDGFRα-Fc proteins (expressed as a percent binding relative to wild-type (WT) PDGFRα) with introduced single glycosylation sites as described in Table 2 or a combination of charge mutations (E52R, L80R, E108R, E111R, L137E, I139E, L208R, M260E, L261R, E263R, K265E).

FIG. 5H is a set of graphs showing biolayer interferometry (BLI) binding curves for the HCMV gHgLgO trimer and PDGFRα-Fc interactions as described in Table 2.

FIG. 6A is a graph showing the level of HCMV trimer (strains: Merlin and VR1814) binding (normalized binding signal of HCMV trimer, expressed as percent of maximum signal) with the indicated human receptor proteins of the Cell-Surface Receptor Discovery Platform results.

FIG. 6B is a schematic diagram showing the domain organization of human TGFβR3. Domains: orphan domain 2 (OD2), orphan domain 1 (OD1), N-terminal zona pellucida domain (ZP-N), C-terminal zona pellucida domain (ZP-C), transmembrane domain (TM), and intracellular domain (ICD).

FIG. 6C is a size exclusion chromatogram showing absorbance at 280 nm for eluted gHgLgO-TGFβR3-13H11-Msl-109 complex (top panel) and a corresponding SDS-PAGE gel image (bottom panel) showing the components of the gHgLgO-TGFβR3-13H11-Msl-109 complex in the indicated SEC fractions. Dotted lines on the chromatogram and beside the SDS-PAGE gel image indicate equivalent SEC elution fractions.

FIG. 6D is a diagram showing the front view of the HCMV gHgLgO trimer complex bound to TGFβR3 OD2. Dark green: TGFβR3. Red: gO subunit. Pink: gL subunit. Blue: gH subunit.

FIG. 6E is a diagram showing the HCMV trimer distal region in a close-up view showing gO, gL and the gH N-terminus with the highlighted surface area (dark green) involved in interaction with TGFβR3 (gray).

FIG. 6F is a diagram showing the results of a conservation analysis of the HCMV gO-gL+gH N-terminus based on sequences from 93 Herpesvirus 5 strains for gO and gH, and 59 Herpesvirus 5 strains for gL. Conservation range: low to high (green to purple).

FIG. 6G is a set of diagrams showing the HCMV trimer distal region in a close-up view showing TGFβR3 OD1-OD2, gO, gL and the gH N terminus. TGFβR3 OD1 is shown with low opacity to illustrate the orientation of the receptor relative to the host-cell membrane. Inset panels show close-up views of key interaction sites (Sites 1-3) between HCMV gHgLgO trimer and TGFβR3. Red: gO subunit. Pink: gL subunit. Blue: gH subunit. Green: TGFβR3 OD2.

FIG. 6H is a set of diagrams showing a structural comparison between the OD2 domains of TGFβR3 and Endoglin (PDB code: 5I04). Right panel shows a close-up view of the TGFβR3 α1 and the corresponding loop region between β6 and β7 of Endoglin.

FIG. 7A is a pair of diagrams showing the binding of PDGFRα (light green) and TGFβR3 (dark green) to HCMV gHgLgO (gray) in a front view (left) and top view (right).

FIG. 7B is a size exclusion chromatogram showing absorbance at 280 nm for eluted gHgLgO-TGFβR3 and gHgLgO-PDGFRα-TGFβR3 complexes (top panel) and corresponding SDS-PAGE gel images (bottom panel) showing the components of the gHgLgO-TGFβR3 and gHgLgO-PDGFRα-TGFβR3 complexes in the indicated SEC fractions.

FIG. 7C is a diagram showing PDGFRα (green) bound to HMCV gHgLgO (red) in a structural comparison and the model of PDGF bound to PDGFRα based on the PDGFB-PDGFRβ co-crystal structure (PDGFB not shown; PDB code: 3MJG).

FIG. 7D is a set of graphs showing BLI binding curves for HCMV gHgLgO trimer having either wild-type gO (Trimer_{WT}) and or mutant gO (Trimer_{MUT}; gO having M84R, F111R, R117E, F136R, R212E, R230E, R234E, R336E, F342E, A351R, and N358R amino acid substitution mutations) contacted with PDGFRα-Fc.

FIG. 7E is a Western blot analysis showing levels of the indicated PDGFRα cellular signaling components in MRC-5 cells. PDGFRα phosphorylation (pY762, pY849) and downstream signaling activity were assessed after addition of the growth factor PDGF-AA in the absence (−) or presence (+) of the HMCV gHgLgO trimer with wild-type or mutant gO (as in FIG. 7D).

FIG. 7F is a schematic diagram showing a working model of receptor binding by the HCMV gHgLgO trimer and neutralization of trimer binding by an antibody.

FIG. 8A is a schematic diagram showing the HCMV gHgLgO purification and reconstitution process with Fabs 13H11 and Msl-109. Histidine (HIS); streptavidin (STREP); size exclusion chromatography (SEC).

FIG. 8B is a size exclusion chromatogram showing absorbance at 280 nm for eluted gHgLgO-13H11-Msl-109 complex (top panel) and a corresponding SDS-PAGE gel image (bottom panel) showing the components of the gHgLgO-13H11-Msl-109 complex in the indicated SEC fractions. Dotted lines on the chromatogram and beside the SDS-PAGE gel image indicate equivalent SEC elution fractions.

FIG. 8C is a cryo-EM micrograph showing the gHgLgO-13H11-Msl-109 complex. Scale bar: 10 nm.

FIG. 8D is a set of cryo-EM micrographs showing representative 2D class averages of monomeric and dimeric gHgLgO-13H11-Msl-109. Scale bar: 10 nm.

FIG. 8E is a schematic diagram of the processing workflow to obtain an ab-initio 3D reconstruction of gHgLgO-13H11-Msl-109.

FIG. 8F is a schematic diagram showing the data collection and processing scheme to obtain a high-resolution 3D reconstruction of gHgLgO-13H11-Msl-109.

FIG. 8G is diagram showing an isosurface rendering of the gHgLgO-13H11-Msl-109 3D map before focused refinement with surface coloring according to the local resolution estimated by windowed Fourier shell correlations (FSCs). Resolution range: 2.7 to >4.7 Å (blue to red).

FIG. 8H is a heat map representation of the distribution of assigned particle orientations. Heat map shows the number of particles arranged in a defined orientation in 3D space.

FIG. 8I is a graph showing the FSC between half data sets for the overall gHgLgO-13H11-Msl-109 3D reconstruction and for the focused refinement reconstructions (as shown in FIG. 8F).

FIG. 9A is a set of ribbon diagrams showing a structural comparison of the HCMV trimer and pentamer (PDB code: 5VOB) gH subunits divided in domains DI, DII, DII and DIV and of the gL subunits.

FIG. 9B is a pair of diagrams showing the interaction interface of gO mapped on gL (top) and of UL130 and UL128 on gL (bottom, based on PDB code: 5VOB).

FIG. 9C is a pair of diagrams showing the glycosylation site distribution (colored molecules) on the HCMV pentamer complex (PDB code: 5VOB) with highlighted putative receptor binding sites.

FIG. 10 is a diagram showing a close-up view of the variable Fab regions for 13H11 and Msl-109 bound to the DII-DIV regions of gH. Fab contact regions on gH are highlighted that were previously identified by hydrogen exchange mass spectrometry.

FIG. 11A is a size exclusion chromatogram showing absorbance at 280 nm for eluted gHgLgO-PDGFRα-13H11-Msl-109 complex (top panel) and a corresponding SDS-PAGE gel image (bottom panel) showing the components of the gHgLgO-PDGFRα-13H11-Msl-109 complex in the indicated SEC fractions. Dotted lines on the chromatogram and beside the SDS-PAGE gel image indicate equivalent SEC elution fractions.

FIG. 11B is a representative cryo-EM micrograph showing the gHgLgO-PDGFRα-13H11-Msl-109 complex.

FIG. 11C is a set of cryo-EM micrographs showing representative 2D class averages of the gHgLgO-PDGFRα-13H11-Msl-109 complex.

FIG. 11D is a schematic diagram showing the data collection and processing scheme to obtain a high-resolution 3D reconstruction of the gHgLgO-PDGFRα-13H11-Msl-109 complex.

FIG. 11E is a diagram showing an isosurface rendering of the gHgLgO-PDGFRα-13H11-Msl-109 3D map before focused refinement with surface coloring according to the local resolution estimated by windowed FSCs.

FIG. 11F is a diagram showing a heat map representation of the distribution of assigned particle orientations. Heat map shows the number of particles arranged in a defined orientation in 3D space.

FIG. 11G is a graph showing the FSC between half data sets for the gHgLgO-PDGFRα-13H11-Msl-109 3D reconstruction and for the focused refinement reconstructions (as shown in FIG. 11D).

FIG. 12A is a set of diagrams showing a structural comparison of D1-D3 of PDGFRα (Trimer bound) and PDGFRβ (PDGFβ not shown; PDB code: 3MJG), Kit (SCF not shown; PDB code: 2E9W) or FMS (M-CSF not shown; PDB code: 3EJJ).

FIG. 12B is a set of ribbon diagrams showing a structural comparison of individual D1, D2 and D3 domains of PDGFRα (Trimer bound) and PDGFRβ (PDGFB not shown; PDB code: 3MJG).

FIG. 12C is a set of ribbon diagrams showing a structural comparison of D1-D3 PDGFRα (Trimer bound) and PDGFRβ (PDGFB not shown; PDB code: 3MJG) after alignment on D2.

FIG. 12D is a stick diagram showing a structural comparison of gHgLgO-PDGFRα and gHgLgO (Fabs 13H11 and Msl-109 not shown).

FIG. 12E is a sequence alignment diagram showing the PDGFRα structure-based sequence alignment of the D1-D3 region to the PDGFRβ sequence. Interaction sites with the HCMV Trimer gHgLgO (Sites 1-4) and to PDGFβ are highlighted in red boxes.

FIG. 13A is a representative cryo-EM micrograph showing the gHgLgO-TGFβR3-13H11-Msl-109 complex. Scale bar: 10 nm.

FIG. 13B is a set of representative cryo-EM micrographs showing 2D class averages of gHgLgO-TGFβR3-13H11-Msl-109 complex. Scale bar: 10 nm.

FIG. 13C is a schematic diagram showing the data collection and processing scheme to obtain a high-resolution 3D reconstruction of gHgLgO-TGFβR3-13H11-Msl-109 complex.

FIG. 13D is a diagram showing the isosurface rendering of the gHgLgO-TGFβR3-13H11-Msl-109 3D map before focused refinement with surface coloring according to the local resolution estimated by windowed FSCs. Resolution range: 2.5 to >5 Å (blue to red).

FIG. 13E is a heat map representation showing the distribution of assigned particle orientations. Heat map shows the number of particles arranged in a defined orientation in 3D space.

FIG. 13F is a graph showing FSC between half data sets for the gHgLgO-TGFβR3-13H11-Msl-109 3D reconstruction and for the focused refinement reconstructions (as shown in FIG. 13E).

FIG. 13G is a diagram showing a structural comparison of gHgLgO-TGFβR3 (green) and gHgLgO (Fabs 13H11 and Msl-109 not shown).

FIG. 13H is a sequence alignment diagram showing TGFβR3 structure-based sequence alignment of OD2 region to the Endoglin OD2 sequence. Interaction sites with the HCMV Trimer gHgLgO (Sites 1-3) are highlighted in red boxes.

FIG. 14A is a pair of size exclusion chromatograms showing absorbance at 280 nm for eluted PDGFRα and TGFβR3 (left panel) and corresponding SDS-PAGE gel images (right panel) showing PDGFRα and TGFβR3 in the indicated SEC fractions.

FIG. 14B is a pair of size exclusion chromatograms showing absorbance at 280 nm for eluted HCMV gHgLgO trimer and gHgLgO-PDGFRα complex (left panel) and corresponding SDS-PAGE gel images (right panel) showing the components of the gHgLgO-PDGFRα complex in the indicated SEC fractions.

FIG. 14C is a pair of size exclusion chromatograms showing absorbance at 280 nm for eluted gHgLgO-TGFβR3 and gHgLgO-PDGFRα-TGFβR3 complexes (left panel) and corresponding SDS-PAGE gel images (right panel) showing the components of the gHgLgO-TGFβR3 and gHgLgO-PDGFRα-TGFβR3 complexes. gHgLgO-TGFβR3 was preincubated with equimolar amounts of PDGFRα.

### DETAILED DESCRIPTION OF THE INVENTION

### 1. Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) aspects that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

The terms “patient,” “subject,” or “individual,” as used interchangeably herein, refer to a human patient.

An “intravenous” or “iv” dose, administration, or formulation of a drug is one which is administered via a vein, e.g. by infusion.

A “subcutaneous” or “sc” dose, administration, or formulation of a drug is one which is administered under the skin, e.g. via a pre-filled syringe, auto-injector, or other device.

For the purposes herein, “clinical status” refers to a patient's health condition. Examples include that the patient is improving or getting worse. In one embodiment, clinical status is based on an ordinal scale of clinical status. In one embodiment, clinical status is not based on whether or not the patient has a fever.

An “effective amount” refers to an amount of an agent (e.g., a therapeutic agent) that is effective to bring about a therapeutic/prophylactic benefit (e.g., as described herein) that is not outweighed by unwanted/undesirable side effects.

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (iv) administration. In another embodiment, the formulation is for subcutaneous (sc) administration.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein any form of the protein that results from processing in the cell. The term also encompasses naturally occurring variants of the protein, e.g., splice variants or allelic variants, e.g., amino acid substitution mutations or amino acid deletion mutations. The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, the terms “human cytomegalovirus (HCMV) trimer”, “HCMV gHgLgO trimer”, and “HCMV trimer” refer to a glycoprotein complex that is located on the outer surface of the viral envelope of human cytomegalovirus (HCMV) and is composed of gH, gL, and gO glycoprotein subunits.

The terms “gO subunit of human cytomegalovirus (HCMV)”, “gO subunit,” and “gO,” as used herein, broadly refer to any native gO from any mammalian source, including primates (e.g. humans) and rodents (e.g., mice and rats), unless otherwise indicated. The term encompasses full-length gO and isolated regions or domains of gO. The term also encompasses naturally occurring variants of gO, e.g., splice variants or allelic variants. The amino acid sequence of an exemplary human gO is provided as SEQ ID NO: 1. Minor sequence variations, especially conservative amino acid substitutions of gO that do not affect gO function and/or activity, are also contemplated by the invention.

The terms “gH subunit of human cytomegalovirus (HCMV)”, “gH subunit,” and “gH,” as used herein, broadly refer to any native gH from any mammalian source, including primates (e.g. humans) and rodents (e.g., mice and rats), unless otherwise indicated. The term encompasses full-length gH and isolated regions or domains of gH. The term also encompasses naturally occurring variants of gH, e.g., splice variants or allelic variants. The amino acid sequence of an exemplary human gH is provided as SEQ ID NO: 2. Minor sequence variations, especially conservative amino acid substitutions of gH that do not affect gH function and/or activity, are also contemplated by the invention.

The terms “gL subunit of human cytomegalovirus (HCMV)”, “gL subunit,” and “gL,” as used herein, broadly refer to any native gL from any mammalian source, including primates (e.g. humans) and rodents (e.g., mice and rats), unless otherwise indicated. The term encompasses full-length gL and isolated regions or domains of gL. The term also encompasses naturally occurring variants of gL, e.g., splice variants or allelic variants. The amino acid sequence of an exemplary human gL is provided as SEQ ID NO: 3. Minor sequence variations, especially conservative amino acid substitutions of gL that do not affect gL function and/or activity, are also contemplated by the invention.

As used herein, a “modulator” is an agent that modulates (e.g., increases, decreases, activates, or inhibits) a given biological activity, e.g., an interaction or a downstream activity resulting from an interaction. A modulator or candidate modulator may be, e.g., a small molecule, an antibody (e.g., a bispecific or multispecific antibody), an antigen-binding fragment (e.g., a bis-Fab, an Fv, a Fab, a Fab′-SH, a F(ab′)₂, a diabody, a linear antibody, an scFv, an ScFab, a VH domain, or a VHH domain), a peptide, a mimic, an antisense oligonucleotide, or an inhibitory nucleic acid (e.g., an antisense oligonucleotide (ASO) or a small interfering RNA (siRNA)).

By “increase” or “activate” is meant the ability to cause an overall increase, for example, of 20% or greater, of 50% or greater, or of 75%, 85%, 90%, or 95% or greater. In certain aspects, increase or activate can refer to a downstream activity of a protein-protein interaction.

By “reduce” or “inhibit” is meant the ability to cause an overall decrease, for example, of 20% or greater, of 50% or greater, or of 75%, 85%, 90%, or 95% or greater. In certain aspects, reduce or inhibit can refer to a downstream activity of a protein-protein interaction.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

The terms “host cell,” “host cell line,” and “host cell culture” are used interchangeably and refer to cells into which exogenous nucleic acid has been introduced, including the progeny of such cells. Host cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the host cell is stably transformed with the exogenous nucleic acid. In other aspects, the host cell is transiently transformed with the exogenous nucleic acid.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antigen-binding fragment” or “antibody fragment” refers to a molecule other than an intact antibody that comprises a portion of an intact antibody that binds the antigen to which the intact antibody binds. Examples of antigen-binding fragments include but are not limited to bis-Fabs; Fv; Fab; Fab, Fab′-SH; F(ab′)₂; diabodies; linear antibodies; single-chain antibody molecules (e.g., scFv, scFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The terms “full-length antibody,” “intact antibody,” and “whole antibody” are used herein interchangeably to refer to an antibody having a structure substantially similar to a native antibody structure or having heavy chains that contain an Fc region as defined herein.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

The term “small molecule” refers to any molecule with a molecular weight of about 2000 daltons or less, e.g., about 1000 daltons or less. In some aspects, the small molecule is a small organic molecule.

The term “mimic” or “molecular mimic,” as used herein, refers to a polypeptide having sufficient similarity in conformation and/or binding ability (e.g., secondary structure, tertiary structure) to a given polypeptide or to a portion of said polypeptide to bind to a binding partner of said polypeptide. The mimic may bind the binding partner with equal, less, or greater affinity than the polypeptide it mimics. A molecular mimic may or may not have obvious amino acid sequence similarity to the polypeptide it mimics. A mimic may be naturally occurring or may be engineered. In some aspects, the mimic is a mimic of a member of a binding pair. In yet other aspects, the mimic is a mimic of another protein that binds to a member of the binding pair. In some aspects, the mimic may perform all functions of the mimicked polypeptide. In other aspects, the mimic does not perform all functions of the mimicked polypeptide.

As used herein, the term “conditions permitting the binding” of two or more proteins to each other refers to conditions (e.g., protein concentration, temperature, pH, salt concentration) under which the two or more proteins would interact in the absence of a modulator or a candidate modulator. Conditions permitting binding may differ for individual proteins and may differ between protein-protein interaction assays (e.g., surface plasmon resonance assays, biolayer interferometry assays, enzyme-linked immunosorbent assays (ELISA), extracellular interaction assays, and cell surface interaction assays.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease (e.g., preventing HCMV infection or symptoms thereof), reducing or preventing secondary infection in a patient having an infection (e.g., reducing or preventing secondary infection of nervous tissue, immune cells, lymphoid tissue, and/or lung tissue), alleviation of symptoms, diminishment of any direct or indirect pathological consequences of the disease, decreasing the rate of disease progression, amelioration or palliation of the disease state, and remission or improved prognosis.

The “pathology” of a disease or condition includes all phenomena that compromise the well-being of the patient.

“Amelioration,” “ameliorating,” “alleviation,” “alleviating,” or equivalents thereof, refers to both therapeutic treatment and prophylactic or preventative measures, wherein the object is to ameliorate, prevent, slow down (lessen), decrease or inhibit a disease or condition, e.g., HCMV infection. Those in need of treatment include those already with the disease or condition as well as those prone to having the disease or condition or those in whom the disease or condition is to be prevented.

### II. Modulators of Protein-Protein Interactions

In some aspects, the disclosure features an isolated modulator of the interaction between PDGFRα or TGFβR3 and the HCMV gHgLgO trimer, wherein the modulator causes a decrease in the binding of the HCMV gHgLgO trimer to PDGFRα or TGFβR3 relative to binding in the absence of the modulator.

A. Modulators of the Interaction Between PDGFRα and the HCMV gHgLgO Trimer

i. Modulators that Bind the HCMV gHgLgO Trimer

In some aspects, the disclosure features a modulator of the interaction between the gO subunit of the human cytomegalovirus (HCMV) gHgLgO trimer and PDGFRα that binds to the glycosylation-free surface of the gO subunit and causes a decrease in the binding of the gO subunit to PDGFRα.

In some aspects, the modulator binds to (a) one or more of residues R230, R234, V235, K237, and Y238 of the gO subunit (e.g., one, two, three, four, or all five of R230, R234, V235, K237, and Y238); (b) one or more of residues N81, L82, M84, M86, F109, F111, T114, Q115, R117, K121, and V123 of the gO subunit (e.g., one, two, three, four, five, six, seven, eight, nine, ten, or all eleven of N81, L82, M84, M86, F109, F111, T114, Q115, R117, K121, and V123); and (c) one or more of residues R336, Y337, K344, D346, N348, E354, and N358 of the gO subunit (e.g., one, two, three, four, five, six, or all seven of R336, Y337, K344, D346, N348, E354, and N358).

In some aspects, the disclosure features a modulator of the interaction between the gO subunit of the HCMV gHgLgO trimer and PDGFRα that binds to (a) one or more of residues R230, R234, V235, K237 and Y238 of the gO subunit (e.g., one, two, three, four, or all five of R230, R234, V235, K237, and Y238); (b) one or more of residues N81, L82, M84, M86, F109, F111, T114, Q115, R117, K121, and V123 of the gO subunit (e.g., one, two, three, four, five, six, seven, eight, nine, ten, or all eleven of N81, L82, M84, M86, F109, F111, T114, Q115, R117, K121, and V123); and (c) one or more of residues R336, Y337, K344, D346, N348, E354, and N358 of the gO subunit (e.g., one, two, three, four, five, six, or all seven of R336, Y337, K344, D346, N348, E354, and N358); and causes a decrease in the binding of the gO subunit to PDGFRα.

In some aspects, the modulator binds to all 23 of residues R230, R234, V235, K237, Y238, N81, L82, M84, M86, F109, F111, T114, Q115, R117, K121, V123, R336, Y337, K344, D346, N348, E354, and N358 of the gO subunit.

In some aspects, the modulator further binds to one or more of residues R47, Y84, and N85 of the gH subunit of HCMV (e.g., one, two, or all three of R47, Y84, and N85). In some aspects, the modulator further causes a decrease in the binding of the gH subunit to PDGFRα.

In some aspects, the modulator is a small molecule, an antibody or antigen-binding fragment thereof, a peptide, a mimic, or an inhibitory nucleic acid (e.g., ASO or a siRNA). Modulators are further described below.

In some aspects, the antibody is a bispecific antibody or a multispecific antibody. In some aspects, the bispecific antibody or multispecific antibody binds to at least three distinct epitopes of the gO subunit. In some aspects, the at least three distinct epitopes comprise (a) a first epitope comprising one or more of residues R230, R234, V235, K237 and Y238 of the gO subunit; (b) a second epitope comprising one or more of residues N81, L82, M84, M86, F109, F111, T114, Q115, R117, K121, and V123 of the gO subunit; and (c) a third epitope comprising one or more of residues R336, Y337, K344, D346, N348, E354, and N358 of the gO subunit.

In some aspects, the modulator is a mimic of PDGFRα.

ii. Modulators that Bind PDGFRα

In some aspects, the disclosure features a modulator of the interaction between the gO subunit of the HCMV gHgLgO trimer and PDGFRα that binds to the D1, D2, and D3 domains of PDGFRα and causes a decrease in the binding of the gO subunit to PDGFRα.

In some aspects, the modulator binds to (a) one or more of residues N103, Q106, T107, E108, and E109 of PDGFRα (e.g., one, two, three, four, or all five of N103, Q106, T107, E108, and E109); (b) one or more of residues M133, L137, I139, E141, I147, S145, Y206, and L208 of PDGFRα (e.g., one, two, three, four, five, six, seven or all eight of M133, L137, I139, E141, I147, S145, Y206, and L208); and (c) one or more of N240, D244, Q246, T259, E263, and K265 of PDGFRα (e.g., one, two, three, four, five, or all six of N240, D244, Q246, T259, E263, and K265).

In some aspects, the disclosure features a modulator of the interaction between the gO subunit of the HCMV gHgLgO trimer and PDGFRα that binds to (a) one or more of residues N103, Q106, T107, E108, and E109 of PDGFRα (e.g., one, two, three, four, or all five of N103, Q106, T107, E108, and E109); (b) one or more of residues M133, L137, I139, E141, I147, S145, Y206, and L208 of PDGFRα (e.g., one, two, three, four, five, six, seven, or all eight of M133, L137, I139, E141, I147, S145, Y206, and L208); and (c) one or more of residues N240, D244, Q246, T259, E263, and K265 of PDGFRα (e.g., one, two, three, four, five, or all six of N240, D244, Q246, T259, E263, and K265) and causes a decrease in the binding of the gO subunit to PDGFRα.

In some aspects, the modulator binds to all nineteen of residues N103, Q106, T107, E108, E109, M133, L137, I139, E141, I147, S145, Y206, L208, N240, D244, Q246, T259, E263, and K265 of PDGFRα.

In some aspects, the modulator further binds to one or more of residues E52, S78, and L80 of PDGFRα. In some aspects, the modulator further causes a decrease in the binding of the gH subunit to PDGFRα.

In some aspects, the modulator is a small molecule, an antibody or antigen-binding fragment thereof, a peptide, a mimic, or an inhibitory nucleic acid (e.g., ASO or a siRNA). Modulators are further described below.

In some aspects, the antibody is a bispecific antibody or a multispecific antibody. In some aspects, the bispecific antibody or multispecific antibody binds to at least three distinct epitopes of PDGFRα. In some aspects, the at least three distinct epitopes comprise (a) a first epitope comprising one or more of residues N103, Q106, T107, E108, and E109 of PDGFRα; (b) a second epitope comprising one or more of residues M133, L137, I139, E141, I147, S145, Y206, and L208 of PDGFRα; and (c) a third epitope comprising one or more of residues N240, D244, Q246, T259, E263, and K265 of PDGFRα.

In some aspects, the modulator is a mimic of the gO subunit of the HCMV gHgLgO trimer.

iii. Reduction in Binding and/or Infection

In some aspects, the modulator decreases binding of the gO subunit of the HCMV gHgLgO trimer to PDGFRα by at least 50%. In some aspects, the decrease in binding is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., binding is abolished), e.g., the decrease is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator decreases binding of the gO subunit of HCMV trimer to PDGFRα by at least 90%. In some aspects, the decrease in binding is at least 50%, e.g., as measured by surface plasmon resonance, biolayer interferometry, or an enzyme-linked immunosorbent assay (ELISA).

In some aspects, the modulator decreases binding of the gO subunit of the HCMV gHgLgO trimer to TGFβR3 by at least 50%. In some aspects, the decrease in binding is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., binding is abolished), e.g., the decrease is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator decreases binding of the gO subunit of HCMV trimer to TGFβR3 by at least 90%. In some aspects, the decrease in binding is at least 50%, e.g., as measured by surface plasmon resonance, biolayer interferometry, or an enzyme-linked immunosorbent assay (ELISA).

In some aspects, the modulator causes a decrease in infection of a cell by HCMV relative to infection in the absence of the modulator. In some aspects, infection is decreased by at least 40%, as measured in a viral infection assay or a viral entry assay using pseudotyped particles. In some aspects, the decrease is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., no infection occurs), e.g., the decrease is 5%-15%, 15%-25%, 25%-35%, 35%-45%, 45%-55%, 55%-65%, 65%-75%, 75%-85%, 85%-95%, or 95%-100%).

In some aspects, the modulator has minimal binding with a region of PDGFRα that triggers downstream signaling or does not bind to a region of PDGFRα that triggers downstream signaling. In some aspects, the region of PDGFRα that triggers downstream signaling is a binding site of PDGF. In some aspects, the modulator does not sterically hinder or causes minimal steric hindrance of binding of a PDGFRα ligand to a region of PDGFRα that triggers downstream signaling, e.g., does not sterically hinder or causes minimal steric hindrance of binding of PDGF to PDGFRα.

In some aspects, the modulator causes less than a 20% decrease in signaling by PDGFRα compared to signaling in the absence of the modulator. In some aspects, the modulator causes less than a 5%, 10%, 15%, 20%, 25%, 30%, 35%, 40%, 45%, 50%, 55%, 60%, 65%, 70%, 75%, 80%, 85%, 90%, or 95% decrease in signaling by PDGFRα compared to signaling in the absence of the modulator (e.g., causes a 0%-5%, 5%-15%, 15%-25%, 25%-35%, 35%-45%, 45%-55%, 55%-65%, 65%-75%, 75%-85%, or 85-95% decrease in signaling by PDGFRα compared to signaling in the absence of the modulator). In some aspects, the modulator does not cause a decrease in signaling by PDGFRα compared to signaling in the absence of the modulator.

In some aspects, the modulator comprises a pharmaceutically acceptable carrier.

B. Modulators of the Interaction Between TGFβR3 and the HCMV gHgLgO Trimer

i. Modulators that Bind the HCMV gHgLgO Trimer

In some aspects, the disclosure features a modulator of the interaction between the HCMV gHgLgO trimer and TGFβR3 that binds to (a) one or more of residues Q115, L116, R117, and K118 of the gO subunit of the HCMV gHgLgO trimer (e.g., one, two, three or all four of Q115, L116, R117, and K118); (b) one or both of residues Y188 and P191 of the gO subunit of the HCMV gHgLgO trimer and residue N97 of the gL subunit of the HCMV trimer; and (c) one or both of residues T92 and E94 of the gL subunit of the HCMV gHgLgO trimer and causes a decrease in the binding of the HCMV gHgLgO trimer to TGFβR3.

In some aspects, the modulator is a small molecule, an antibody or antigen-binding fragment thereof, a peptide, a mimic, or an inhibitory nucleic acid (e.g., ASO or a siRNA). In some aspects, the antibody is a bispecific antibody or a multispecific antibody.

ii. Modulators that bind TGFβR3

In some aspects, the disclosure features a modulator of the interaction between the HCMV gHgLgO trimer and TGFβR3 that binds to (a) one or more of residues V135, Q136, F137, and S143 of TGFβR3 (e.g., one, two, three or all four of V135, Q136, F137, and S143); (b) one or more of residues R151, N152, and E167 of TGFβR3; and (c) one or both of residues W163 and K166 of TGFβR3 and causes a decrease in the binding of the HCMV gHgLgO trimer to TGFβR3.

In some aspects, the modulator is a small molecule, an antibody or antigen-binding fragment thereof, a peptide, a mimic, or an inhibitory nucleic acid (e.g., ASO or a siRNA). In some aspects, the antibody is a bispecific antibody or a multispecific antibody.

iii. Reduction in Binding and/or Infection

In some aspects, the modulator decreases binding of the gO subunit of the HCMV gHgLgO trimer to TGFβR3 by at least 50%. In some aspects, the decrease in binding is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., binding is abolished), e.g., the decrease is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator decreases binding of the gO subunit of HCMV trimer to TGFβR3 by at least 90%. In some aspects, the decrease in binding is at least 50%, e.g., as measured by surface plasmon resonance, biolayer interferometry, or an enzyme-linked immunosorbent assay (ELISA).

In some aspects, the modulator decreases binding of the gO subunit of the HCMV gHgLgO trimer to PDGFRα by at least 50%. In some aspects, the decrease in binding is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., binding is abolished), e.g., the decrease is 5%-15%, 15%-25%, 25%-35%, 35%-45%, 45%-55%, 55%-65%, 65%-75%, 75%-85%, 85%-95%, or 95%-100%, relative to binding in the absence of the modulator. In some aspects, the modulator decreases binding of the gO subunit of HCMV trimer to PDGFRα by at least 90%. In some aspects, the decrease in binding is at least 50%, e.g., as measured by surface plasmon resonance, biolayer interferometry, or an enzyme-linked immunosorbent assay (ELISA).

In some aspects, the modulator causes a decrease in infection of a cell by HCMV relative to infection in the absence of the modulator. In some aspects, infection is decreased by at least 40%, as measured in a viral infection assay or a viral entry assay using pseudotyped particles. In some aspects, the decrease is at least 5%, 10%, 15%, 20%, 25%, 30%, 35%, 40%, 45%, 50%, 55%, 60%, 65%, 70%, 75%, 80%, 85%, 90%, 95%, 98%, or 99% or is 100% (i.e., no infection occurs), e.g., the decrease is 5%-15%, 15%-25%, 25%-35%, 35%-45%, 45%-55%, 55%-65%, 65%-75%, 75%-85%, 85%-95%, or 95%-100%).

In some aspects, the modulator comprises a pharmaceutically acceptable carrier.

C. Small Molecules

In some aspects, the modulator or candidate modulator is a small molecule. Small molecules are molecules other than binding polypeptides or antibodies as defined herein that may bind, preferably specifically, to PDGFRα (e.g., the D1, D2, and/or D3 domains thereof), TGFβR3, or the HCMV gHgLgO trimer (e.g., gO and/or gH). Binding small molecules may be identified and chemically synthesized using known methodology (see, e.g., PCT Publication Nos. WO00/00823 and WO00/39585). Binding small molecules are usually less than about 2000 daltons in size (e.g., less than about 2000, 1500, 750, 500, 250 or 200 daltons in size), wherein such organic small molecules that are capable of binding, preferably specifically, to a polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). Binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some aspects, the binding of PDGFRα and/or TGFβR3 to the HCMV gHgLgO trimer is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule. In some aspects, the binding of PDGFRα and/or TGFβR3 to the HCMV gHgLgO trimer is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the small molecule. In some aspects, a downstream activity of PDGFRα, TGFβR3, and/or the HCMV gHgLgO trimer (e.g., infection of a cell by HCMV) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule.

D. Antibodies and Antigen-Binding Fragments

In some aspects, the modulator or candidate modulator is an antibody or an antigen-binding fragment thereof binding PDGFRα, TGFβR3, and/or the HCMV gHgLgO trimer. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the modulator is a multispecific antibody, e.g., a bispecific antibody. In some aspects, the modulator is a bispecific or multispecific antibody that binds multiple epitopes of the HCMV gHgLgO trimer, multiple epitopes of PDGFRα, or multiple epitopes of TGFβR3. In some aspects, the modulator is a bispecific or multispecific antibody that binds two or all three of the HCMV gHgLgO trimer, PDGFRα, and TGFβR3

In some aspects, the binding of PDGFRα and/or TGFβR3 to the HCMV gHgLgO trimer is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment. In some aspects, the binding of PDGFRα and/or TGFβR3 to the HCMV gHgLgO trimer is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the antibody or antigen-binding fragment. In some aspects, a downstream activity of PDGFRα, TGFβR3, and/or the HCMV gHgLgO trimer (e.g., infection of a cell by HCMV) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment.

E. Peptides

In some aspects, the modulator or candidate modulator is a peptide that binds to PDGFRα, TGFβR3, and/or the HCMV gHgLgO trimer. The peptide may be the peptide may be naturally occurring or may be engineered. In some aspects, the peptide is a fragment of PDGFRα (e.g., the D1, D2, and/or D3 domains thereof), TGFβR3, or the HCMV gHgLgO trimer (e.g., gO and/or gH), or another protein that binds to PDGFRα, TGFβR3, and/or the HCMV gHgLgO trimer. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of PDGFRα and/or TGFβR3 to the HCMV gHgLgO trimer is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide. In some aspects, the binding of PDGFRα and/or TGFβR3 to the HCMV gHgLgO trimer is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the peptide. In some aspects, a downstream activity of PDGFRα, TGFβR3, and/or the HCMV gHgLgO trimer (e.g., infection of a cell by HCMV) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide.

F. Mimics

In some aspects, the modulator or candidate modulator is a mimic, e.g., a molecular mimic, that binds to PDGFRα, TGFβR3, or the HCMV gHgLgO trimer (e.g., gO and/or gH). The mimic may be a molecular mimic of the PDGFRα (e.g., the D1, D2, and/or D3 domains thereof), TGFβR3, or the HCMV gHgLgO trimer (e.g., gO and/or gH), or another protein that binds to PDGFRα, TGFβR3, or the HCMV gHgLgO trimer (e.g., gO and/or gH). In some aspects, the mimic may perform all functions of the mimicked polypeptide. In other aspects, the mimic does not perform all functions of the mimicked polypeptide.

In some aspects, the binding of PDGFRα and/or TGFβR3 to the HCMV gHgLgO trimer is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic. In some aspects, the binding of PDGFRα and/or TGFβR3 to the HCMV gHgLgO trimer is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the mimic. In some aspects, a downstream activity of PDGFRα, TGFβR3, and/or the HCMV gHgLgO trimer (e.g., infection of a cell by HCMV) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic.

G. Assays for Modulation of Protein-Protein Interactions

In some aspects, the binding of PDGFRα or TGFβR3 and the HCMV gHgLgO trimer in the presence or absence of the candidate modulator is assessed in an assay for protein-protein interaction. Modulation of the interaction may be identified as an increase in protein-protein interaction in the presence of the modulator compared to protein-protein interaction in the absence of the modulator, e.g., an increase of 5%, 10%, 15%, 20%, 25%, 30%, 35%, 40%, 45%, 50%, 55%, 60%, 65%, 70%, 80%, 90%, 95%, 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%) in protein-protein interaction. Alternatively, modulation may be identified as a decrease in protein-protein interaction in the presence of the modulator compared to protein-protein interaction in the absence of the modulator, e.g., an decrease of 5%, 10%, 15%, 20%, 25%, 30%, 35%, 40%, 45%, 50%, 55%, 60%, 65%, 70%, 80%, 90%, 95%, or 100% (e.g., 5%-15%, 15%-25%, 25%-35%, 35%-45%, 45%-55%, 55%-65%, 65%-75%, 75%-85%, 85%-95%, or 95%-100%) in protein-protein interaction. The assay for protein-protein interaction may be, e.g., an SPR assay, a biolayer interferometry (BLI) assay, an enzyme-linked immunosorbent assay (ELISA), an extracellular interaction assay, or a cell surface interaction assay.

Exemplary methods for identifying modulators of protein-protein interactions, as well as agents that may modulate such interactions, are described in PCT/US2020/025471, which is hereby incorporated by reference in its entirety.

### IV. Methods of Treating or Preventing HCMV Infections

A. Methods of Treating Individuals Having HCMV Infections

In some aspects, the disclosure features a method for treating an HCMV infection in an individual, the method comprising administering to the individual an effective amount of a modulator described herein (e.g., a modulator of the interaction between the gO subunit of the HCMV gHgLgO trimer and PDGFRα and/or a modulator of the interaction between the HCMV gHgLgO trimer and TGFβR3), thereby treating the individual. In some aspects, the individual is immunocompromised, is pregnant, or is an infant.

In some aspects, the duration or severity of HCMV infection is decreased by at least 40% relative to an individual who has not been administered the modulator. In some aspects, the duration or severity of HCMV infection is decreased by at least 5%, 10%, 15%, 20%, 25%, 30%, 35%, 40%, 45%, 50%, 55%, 60%, 65%, 70%, 80%, 90%, 95%, or 100% (e.g., 5%-15%, 15%-25%, 25%-35%, 35%-45%, 45%-55%, 55%-65%, 65%-75%, 75%-85%, 85%-95%, or 95%-100%).

B. Methods of Preventing HCMV Infection or Secondary Infection

In some aspects, the disclosure features a method for preventing an HCMV infection in an individual, the method comprising administering to the individual an effective amount of a modulator described herein (e.g., a modulator of the interaction between the gO subunit of the HCMV gHgLgO trimer and PDGFRα and/or a modulator of the interaction between the HCMV gHgLgO trimer and TGFβR3), thereby preventing an HCMV infection in the individual.

In some aspects, the modulator decreases the likelihood of a HCMV infection in the individual relative to infection in the absence of the modulator. In some aspects, the likelihood, extent, or severity of HCMV infection is decreased in patients treated according to the above-described methods relative to untreated patients or relative to patients treated using a control method (e.g., SOC), e.g., decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% (e.g., decreased by 5%-15%, 15%-25%, 25%-35%, 35%-45%, 45%-55%, 55%-65%, 65%-75%, 75%-85%, 85%-95%, or 95%-100%).

In some aspects, the disclosure features a method of prophylaxis against a secondary HCMV infection in an individual (e.g., an individual having an HCMV infection), the method comprising administering to the individual an effective amount of a modulator described herein (e.g., a modulator of the interaction between the gO subunit of the HCMV gHgLgO trimer and PDGFRα and/or a modulator of the interaction between the HCMV gHgLgO trimer and TGFβR3), thereby preventing a secondary HCMV infection in the individual. In some aspects, the secondary infection is an infection by HCMV of an uninfected tissue.

In some aspects, the modulator decreases the likelihood of a secondary HCMV infection in the individual relative to secondary infection in the absence of the modulator. In some aspects, the likelihood, extent, or severity of secondary HCMV infection is decreased in patients treated according to the above-described methods relative to untreated patients or relative to patients treated using a control method (e.g., SOC), e.g., decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% (e.g., decreased by 5%-15%, 15%-25%, 25%-35%, 35%-45%, 45%-55%, 55%-65%, 65%-75%, 75%-85%, 85%-95%, or 95%-100%).

C. Combination Therapies

In some aspects of the above-described methods of treatment and prophylaxis, the method comprises administering to the individual at least one additional therapy (e.g., one, two, three, four, or more than four additional therapies). The modulator of the interaction between PDGFRα or TGFβR3 and the HCMV gHgLgO trimer may be administered to the individual prior to, concurrently with, or after the at least one additional therapy.

D. Methods of Delivery

The compositions utilized in the methods described herein (e.g., a modulator of an interaction between PDGFRα or TGFβR3 and the HCMV gHgLgO trimer, e.g., a small molecule, an antibody, an antigen-binding fragment, a peptide, a mimic, an antisense oligonucleotide, or an siRNA) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, a modulator of a protein-protein interaction is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A modulator of a protein-protein interaction described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with and/or administered concurrently with one or more agents currently used to prevent or treat the disorder in question. The effective amount of such other agents depends on the amount of the modulator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

All patent, patent publication and literature references cited in the present specification are hereby incorporated by reference in their entirety.

### V. Examples

### Example 1. Structure of HCMV Trimer gHgLgO

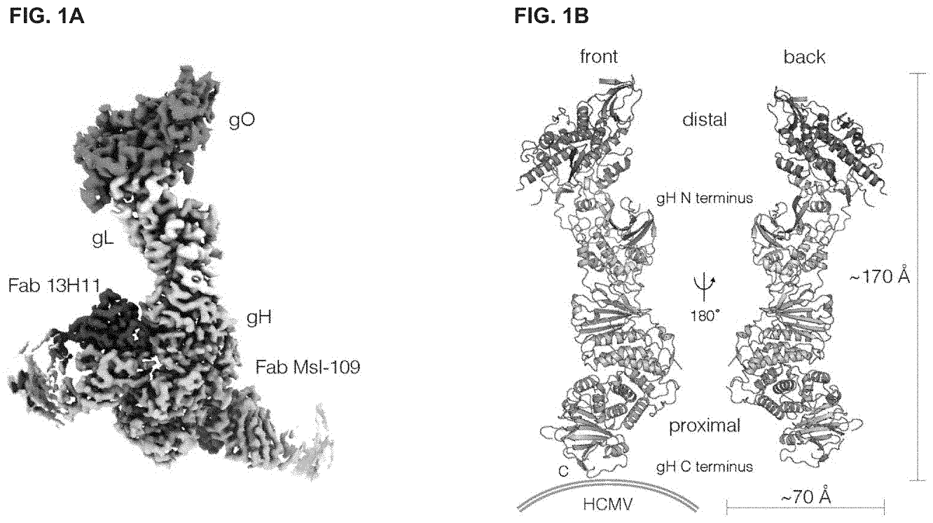

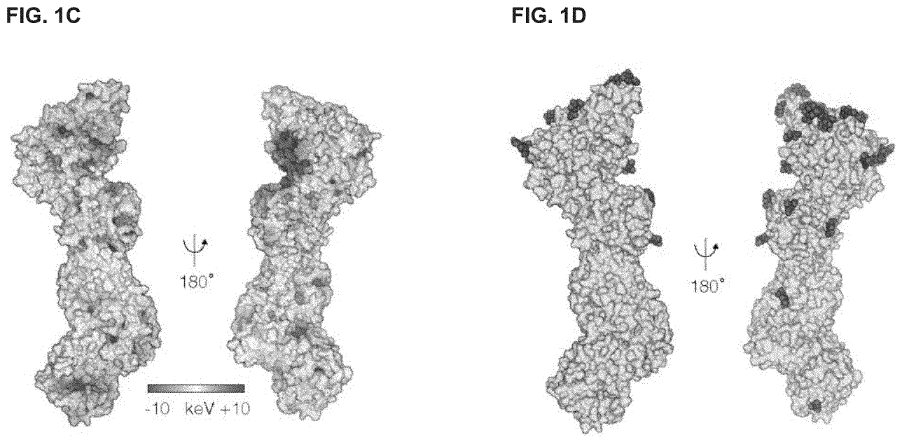

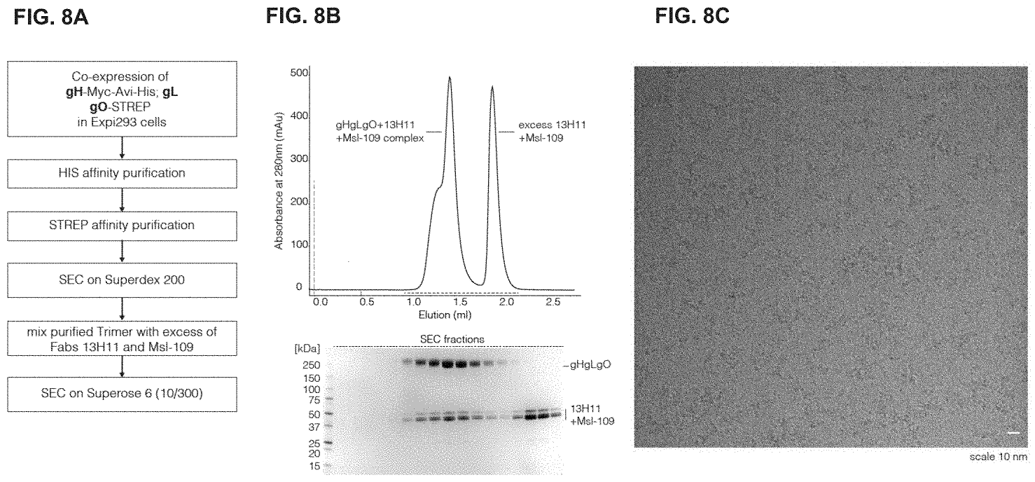

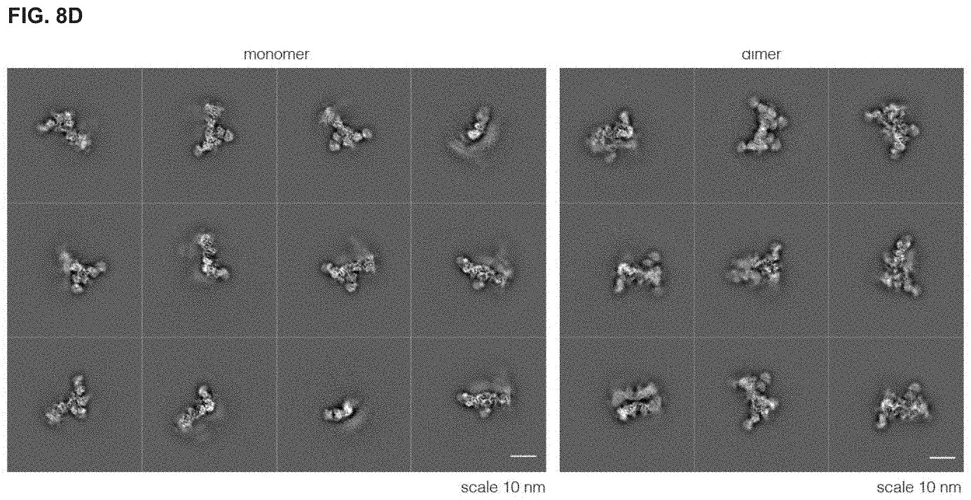

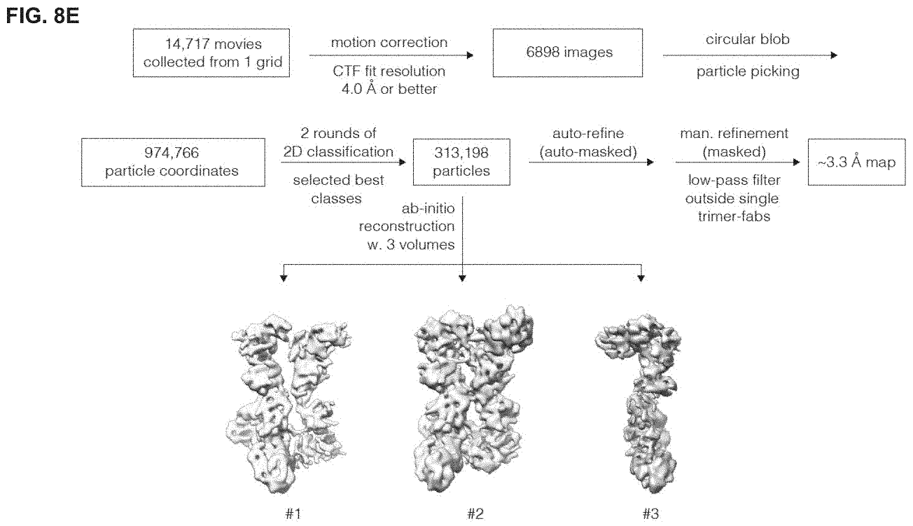

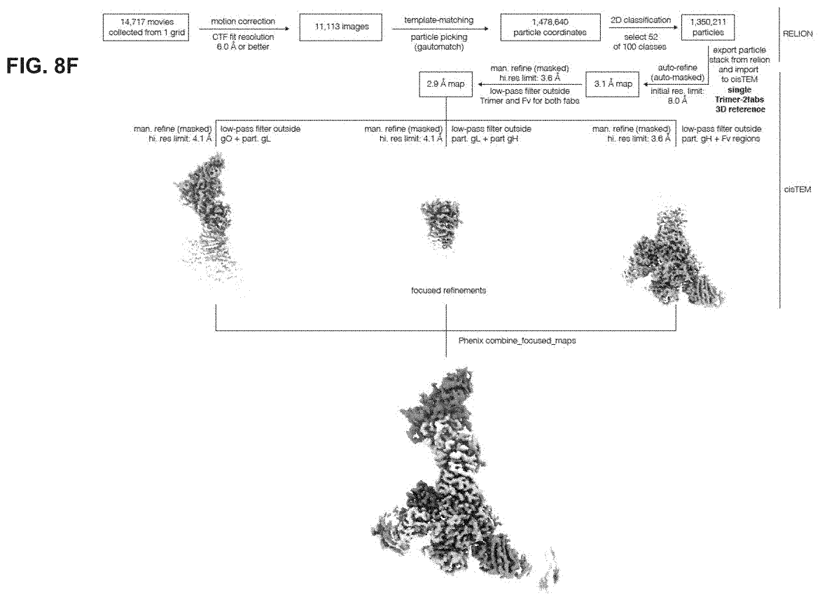

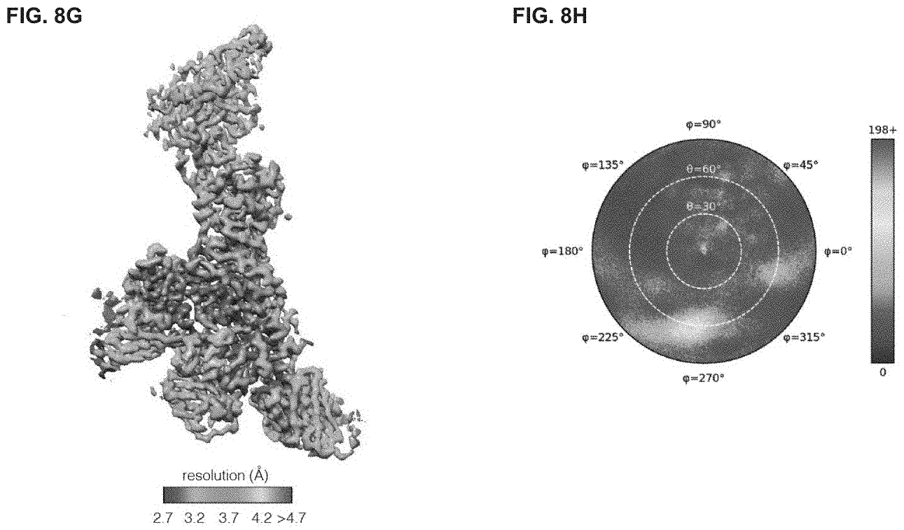

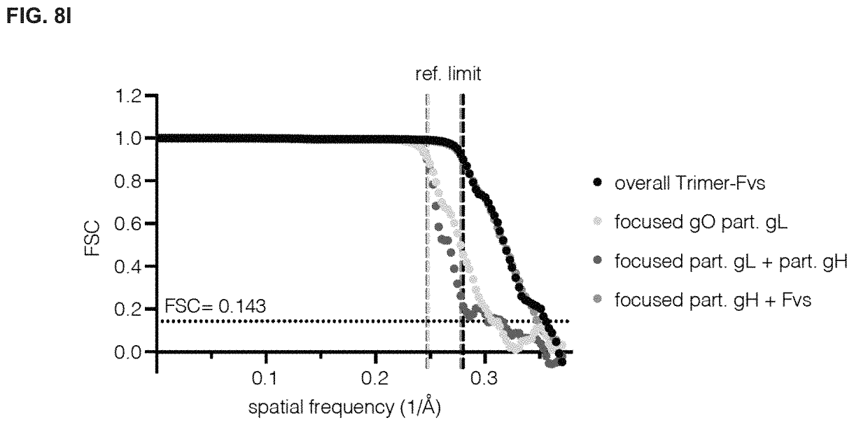

HCMV gHgLgO trimer structural characterization has proven challenging in the past due to its flexibility, elongated nature, and its numerous glycosylation sites, likely hampering the formation of crystals that diffract to high-resolution (Ciferri et al., Proc Natl Acad Sci USA, 112: 1767-1772, 2015). To determine the high-resolution structure of the HCMV trimer, also called the gHgLgO complex, the soluble region of gHgLgO complex was recombinantly expressed in Expi293 cells, and the complex was purified to high purity (FIG. 8A). Consistent with previous reports, HCMV gH, gL and gO were covalently linked by disulfide bonds and run as a single band by SDS-PAGE (FIG. 8B) (Ciferri et al., Proc Natl Acad Sci USA, 112: 1767-1772, 2015). To overcome limitations due to the size, shape and flexibility of the gHgLgO complex, we turned to cryoEM and single particle analysis after reconstituting gHgLgO with the fragment antigen-binding (Fab) regions of the gH-specific neutralizing monoclonal antibodies (mAbs) 13H11 and Msl-109 (FIGS. 8A-8I) (Macagno et al., J Virol, 84: 1005-1013, 2010; Fouts et al., Proc Natl Acad Sci USA, 111: 8209-8214, 2014). Binding of both Fabs increased the size, stability and dimensions of the gHgLgO complex and facilitated high-resolution cryoEM investigation (FIGS. 8C and 8D). The 2D class averages showed a high degree of detail including secondary structure features and particle alignments in different orientations (FIG. 8D). Additionally, a subpopulation of dimeric gHgLgO particles in the 2D classes and 3D ab-initio volumes was noticed (FIGS. 8D and 8E). The dimeric gHgLgO particles orientated head-to-tail in opposing directions with only a few and small contact regions at the interface. The head-to-tail orientation suggests a positioning of the viral membrane at opposing ends of the complex, which seems therefore unlikely to be of physiological relevance. By using a mask around the monomeric gHgLgO complex, a high-resolution structure of the gHgLgO-13H11-Msl-109 complex that extended to a resolution of ˜2.9 Å was determined (FIGS. 1A and 8F-8I and Table 1). The complex was divided into three sub-regions to further improve the map quality across the entire gHgLgO-13H11-Msl-109 molecule using specific masks in focused local refinements (FIG. 8F). The combination of the focused 3D reconstructions allowed building the structure and assigning the sequence to the majority parts of the gH, gL and the previously unknown gO subunits as well as the variable domains (Fv) of both Fabs (FIGS. 1A and 1B).

TABLE 1
Cryo-EM data collection, refinement and validation statistics

| | | |
|---|---|---|
| HCMV Trimer | HCMV Trimer | HCMV Trimer |
| gHgLgO | gHgLgO-hPDGFRα | gHgLgO-hTGFβR3 |

Data Collection

| | | | |
|---|---|---|---|
| Magnification | 165,000x | 165,000x | 165,000x |
| Voltage (kV) | 300 | 300 | 300 |
| Electron exposure (e/Å²) | 48.579 | 49.967 | 53.437 |
| Defocus range (μm) | 0.5-1.5 | 0.5-1.5 | 0.5-1.6 |
| Pixel size (Å) | 0.824 | 0.824 | 0.824 |
| Symmetry imposed | C1 | C1 | C1 |
| Initial particle images | 1,478,640 | 4,151,085 | 2,780,519 |
| Final particle images | 1,350,211 | 3,560,620 | 2,737,199 |
| Map resolution (Å) | 2.9 | 2.8 | 2.6 |
| FSC threshold | 0.143 | 0.143 | 0.143 |
| Map resolution range (Å) | 2.8-54.0 | 2.7-54.0 | 2.5-47.6 |

Refinement

| | | | |
|---|---|---|---|
| Initial models used (PDB | Ab-initio for | Ab-initio for | Ab-initio for |
| code) | gOgHgL: 5VOB | hPDGFRα | hTGFβR3 |
| Model resolution (Å) | 3.2 | 3.1 | 2.8 |
| FSC threshold | 0.5 | 0.5 | 0.5 |
| Model resolution range (Å) | 2.8-54.0 | 2.7-54.0 | 2.5-47.6 |
| Map-sharpening B-factors | −90 | −90 | −90 |
| (Å²) | | | |

Model composition

| | | | |
|---|---|---|---|
| Non-hydrogen atoms | 13549 | 15706 | 14620 |
| Protein residues | 1658 | 1929 | 1793 |
| Waters | 0 | 0 | 0 |
| Ligands | 27 | 29 | 26 |

B factors (Å²)

| | | | |
|---|---|---|---|
| Protein | 82.77 | 61.40 | 34.35 |
| Ligand | 98.07 | 76.66 | 63.22 |

R.m.s. deviations

| | | | |
|---|---|---|---|
| Bond lengths (Å) | 0.003 | 0.002 | 0.003 |
| Bond angles (º) | 0.547 | 0.518 | 0.554 |

Validation

| | | | |
|---|---|---|---|
| MolProbity score | 1.61 | 1.48 | 1.51 |
| Clashscore | 6.36 | 5.98 | 5.86 |
| Poor rotamers (%) | 0.27 | 0.17 | 0.06 |

Ramachandran Plot

| | | | |
|---|---|---|---|
| Favored (%) | 96.20 | 97.15 | 96.9 |
| Allowed (%) | 3.8 | 2.85 | 3.1 |
| Disallowed (%) | 0 | 0 | 0.0 |

The overall structure of the HCMV trimer gHgLgO adopts a boot-like architecture with relative dimensions of 170 Å in length and 70 Å in width (FIG. 1B). The three subunits interact in a linear order, where the C terminus of gH, orients most proximal to the HCMV viral membrane and gO points to the distal end of the molecule for receptor binding (FIGS. 1A and 1B). The gL subunit bridges the gH and gO subunits in the center of the complex (FIGS. 1A and 1B). The electrostatic surface charge is distributed asymmetrically across the gHgLgO complex with a negative charge cluster at the proximal gH region and a positive charge cluster at the distal gO region of the complex (FIG. 1C). Similarly, the 22 observed N-linked glycosylation sites are distributed asymmetrically along the gHgLgO complex with 5 on gH, 1 on gL, and 16 on gO. Remarkably, there is an enrichment of glycosylated residues at the distal gO region and, in particular along the back side of the entire complex (FIG. 1D). In contrast, the front side of the trimer complex appears to lack glycosylated residues in all three subunits (FIG. 1D). Such asymmetric distribution of surface charge and glycosylation have important implications for the receptor interactions and potentially interactions with the prefusion conformation of gB, and therefore could be used to inform design of anti-viral strategies.

Protein Expression and Purification

Optimized coding DNAs for human herpesvirus 5, gH (1-716), gL, and gO (HCMV strain Merlin for gH and gL and strain VR1814 for gO) were each cloned into a pRK vector behind a CMV promoter. A C-terminal Myc-Avi-His tag was added to gH and a C-terminal Twin-Strep-tag was added to gO.

Expi293 cells in suspension were cultured in SMM 293T-I medium under 5% CO₂ at 37° C. and transfected using polyethylenimine (PEI) with DNAs at a 1:1:1 ratio for the gHgLgO expression when the cell density reached 4×10⁶ cells per ml. Transfected cells were cultured for 7 days before harvesting of the expression supernatant.

The HCMV trimer gHgLgO was purified as follows. The expression supernatant corresponding to a 35 l expression volume was concentrated via tangential flow filtration (TFF) to a volume of 1-2 l, loaded on a 20 ml Ni Sepharose Excel (cytiva) resin, washed with 13 column volumes (CV) wash buffer (30 mM TRIS (pH 8.0), 250 mM NaCl, 5% glycerol, 20 mM imidazole) and eluted in 5 CV elution buffer (30 mM TRIS (pH 8.0), 250 mM NaCl, 5% glycerol, 400 mM imidazole). The eluent was applied to 3 ml Strep-Tactin XT high affinity resin (IBA) and bound for 2 h. The resin was washed with 10 CV Strep-wash buffer (25 mM HEPES (pH 7.5), 300 mM NaCl, 5% glycerol) and eluted from the beads in Strep-wash buffer supplemented with 50 mM biotin. The eluate was concentrated with an AMICON® Ultra centrifugal filter device (30 kDa molecular weight cut-off (MWCO)) and loaded on a Superdex 200 10/300 or 10/60 column equilibrated in trimer-SEC buffer (25 mM HEPES (pH 7.5), 300 mM NaCl, 5% glycerol).

The heavy and light chain of Fab Msl-109 were co-expressed under a phoA promoter in E. coli 34B8 cells in phosphate limiting media (C.R.A.P) for 20 h at 30° C. The pellet from a 1-L expression volume was resuspended in 70 ml lysis buffer (1×PBS, 25 mM EDTA) supplemented with Roche protease inhibitor tablets and lysed by sonication. The lysate was cleared by centrifugation at 25,000×g for 1 h and subsequently passed through a 0.45 μm filter. The cleared lysate was loaded onto a 5 ml HiTrap Protein G HP (cytiva) column that was equilibrated in lysis buffer. The column was washed with 10-20 CV lysis buffer and eluted in 0.58% (v/v) acetic acid. The pH of the eluate was immediately adjusted by addition of SP-A buffer (20 mM MES, pH 5.5) and loaded onto a 5 ml HiTrap SP HP cation exchange chromatography column (cytiva). The Fab was eluted in a linear 20 CV gradient to SP-B buffer (20 mM MES (pH 5.5), 500 mM NaCl). The eluate was concentrated using an AMICON® Ultra Centrifugal filter device (10 kDa MWCO) and further purified on a Superdex 200 10/300 column equilibrated in Fab-S200 buffer (25 mM Tris (pH 7.5), 300 mM NaCl). The purified Fab was concentrated an AMICON® Ultra centrifugal filter device (10 kDa MWCO), frozen in liquid nitrogen and stored at −80° C. 13H11 antibody was purified as previously described (Ciferri et al., PLoS Pathog, 11: e1005230, 2015).

Reconstitution of HCMV gHgLgO Trimer with Human Receptor Proteins and Neutralizing Fabs

The gHgLgO-13H11-Msl-109 complex was assembled by incubation of 18.3 μM gHgLgO (300 μg) with an excess of the Fabs 13H11 at 30 μM (150 μg) and Msl-109 at 30 μM (150 μg) for 30 min on ice. The excess of the Fabs was removed by purification on a Superose 6 3.2/300 column equilibrated in SEC-reconst-1 buffer (25 mM HEPES (pH 7.5), 200 mM NaCl). The main peak fraction of gHgLgO-13H11-Msl-109 was diluted with SEC-reconst-1 buffer to a concentration of 0.4 mg/ml for cryo-EM sample preparation.

Cryo-EM Sample Preparation and Data Acquisition

The gHgLgO-13H11-Msl-109 complex was prepared as described in the following manner. Holey carbon grids (C-Flat 45 nm R 1.2/1.3 300 mesh coated with Au/Pd 80/20; Protochips) were glow-discharged for 10 s using the Solarus™ plasma cleaner (Gatan). The complex was gently cross-linked with 0.025% EM-grade glutaraldehyde for 10 min at room temperature and quenched with 9 mM Tris, pH 7.5. 3 μl of the sample (now at about 0.4 mg/ml) was applied to the grid. Grids were blotted with a Vitrobot Mark IV (Thermo Fisher) using 2.5-s blotting time with 100% humidity and plunge-frozen in liquid ethane cooled by liquid nitrogen.

Movie stacks were collected using SerialEM (Mastronarde et al., J Struct Biol. October;152(1):36-51, 2005) on a Titan Krios operated at 300 keV with bioquantum energy filter equipped with a K2 Summit direct electron detector camera (Gatan). Images were recorded at 165,000× magnification corresponding to 0.824 Å per pixel, using a 20 eV energy slit. Each image stack contains 50 frames recorded every 0.2 s for an accumulated dose of ˜50 e Å⁻² and a total exposure time of 10 s. Images were recorded with a set defocus range of 0.5 to 1.5 μm.

Cryo-EM Data Processing

Cryo-EM data were processed using a combination of RELION (Scheres, J Struct Biol., 180(3): 519-30, 2012) and cisTEM (Grant et al., Elife, 7(7): e35383, 2018) software packages.

For the gHgLgO-13H11-Msl-109 complex, a total of 14,717 movies were corrected for frame motion using the MotionCor2 (Zheng et al., Nat Methods, 14(4): 331-332, 2017) implementation in RELION and contrast-transfer function parameters were fit using the 30-4.5 Å band of the spectrum with CTFFIND-4 (Rohou and Grigorieff, J Struct Biol., 192(2): 216-2, 2015). For the generation of the first ab-initio 3D reconstruction, images were filtered on the basis of the detected fit resolution better than 4 Å. A total of 974,766 particles were picked using the circular blob picking tool within cisTEM. Particle were sorted in 2 rounds of cisTEM 2D classification to select the best aligning particles yielding 313,196 particles. These particles were subjected to an ab-initio generation within cisTEM with three target volumes. The volume corresponding to a single HCMV trimer was used as a reference for cisTEM auto-refine and manual refinement with a mask around a single (monomeric) gHgLgO-13H11-Msl-109 complex and by applying low-pass filter (LPF) outside the mask. This map was used as a 3D reference for the high-resolution 3D refinements.

For the generation of a high-resolution 3D reconstruction of the gHgLgO-13H11-Msl-109 complex, CTF fitted images were filtered on the basis of the detected fit resolution better than 6 Å. A total of 1,478,640 particles were picked by template-matching with gautomatch (MRC Laboratory of Molecular Biology) using a 30-A low-pass filtered gHgLgO-13H11-Msl-109 complex reference structure. Particles were sorted during RELION 2D classification and 1,350,211 selected particles were imported into cisTEM for 3D refinements. The gHgLgO-13H11-Msl-109 3D reconstruction was obtained after auto-refine and manual refinements with a mask around a single (monomeric) gHgLgO-13H11-Msl-109 complex and by applying low-pass filter (LPF) outside the mask (filter resolution 20 Å) and a score threshold of 0.25. The outside weight was thereby incrementally reduced from 0.5 to 0.15 in iterative rounds of manual refinements. The 3D reconstructions converged to a map resolution of 2.9 Å (Fourier shell correlation (FSC)=0.143, determined in cisTEM). To improve the quality of the map, focussed refinements were obtained after dividing the map into three distinct regions using masks and of the manual refinements low-pass filter (LPF) outside the mask as described above. The focussed maps were sharpened in cisTEM with the following parameters: flattening from a resolution of 8 Å, applying a pre-cut-off B-factor of ˜90 Å² from the origin of reciprocal space and applying a figure-of-merit filter (Rosenthal and Henderson, J Mol Biol., 333(4):721-45, 2003). For model building and figure preparation, a composite map was generated from the three individual focussed 3D maps using phenix combine_focused_maps.

Model Building and Structure Analysis

The gH and gL subunits of the HCMV pentamer structure (Chandramouli et al., Sci Immunol, 2: eaan1457, 2017) were fit as a rigid body into the cryo-EM map. The gO subunit was built de-novo into the high-resolution cryo-EM map. The resulting model was fit as a rigid body into the cryo-EM map. After extensive rebuilding and manual adjustments, multiple rounds of real space refinement using the phenix.real_space_refinement (Afonine et al., Acta Crystallogr D Struct Biol, 74(Pt 9): 814-840, 2018) tool was used to correct global structural differences between the initial model and the map. The model was further manually adjusted in Coot (Emsley et al., Acta Crystallogr D Biol Crystallogr., 66(Pt 4): 486-501, 2010) through iterative rounds of model building and real space refinements in phenix. The model was validated using phenix.validation_cryoem (Afonine et al., Acta Crystallogr D Struct Biol., 74(Pt 9): 814-840, 2018) with built-in MolProbity scoring (Williams et al., Protein Sci., 27(1): 293-315, 2018). Figures were made using PyMOL (The PyMOL Molecular Graphics System, v.2.07 Schrödinger, LLC), UCSF ChimeraX (Goddard et al., Protein Sci., 27(1): 14-25, 2018). 3D homology structural analysis was performed using the DALI server (Holm, Methods Mol Biol., 2112: 29-42, 2020). Sequences were aligned using Clustal Omega (Sievers et al., Mol Syst Biol., 7: 539, 2011) within JalView (Waterhouse et al., Bioinformatics, 25(9): 1189-91, 2009) and illustrated with ESPript 3.0 (Robert and Gouet, Nucleic Acids Res., 42(Web Server issue): W320-4, 2014) followed by manual adjustment based on considerations from the PDGFRα-gHgLgO-13H11-Msl-109 or the TGFβR3-gHgLgO-13H11-Msl-109 structure models.

### Example 2. Structural Basis for the Assembly of HCMV Trimer and Pentamer Specific Subunits

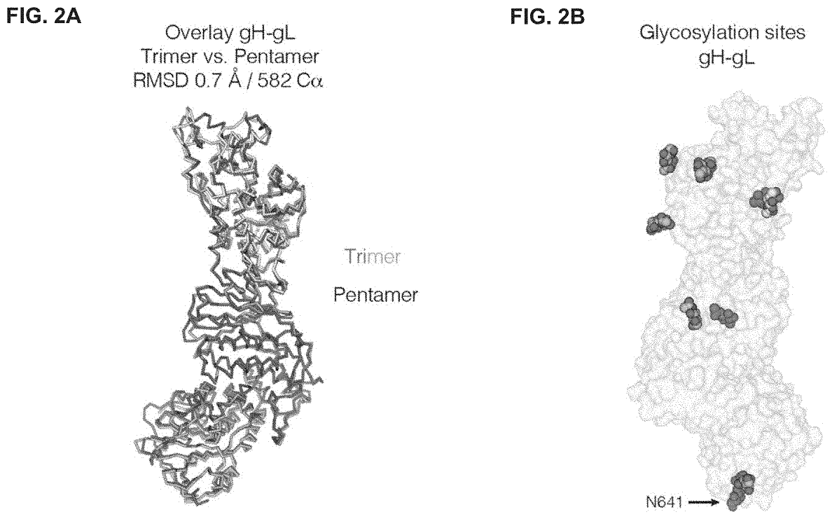

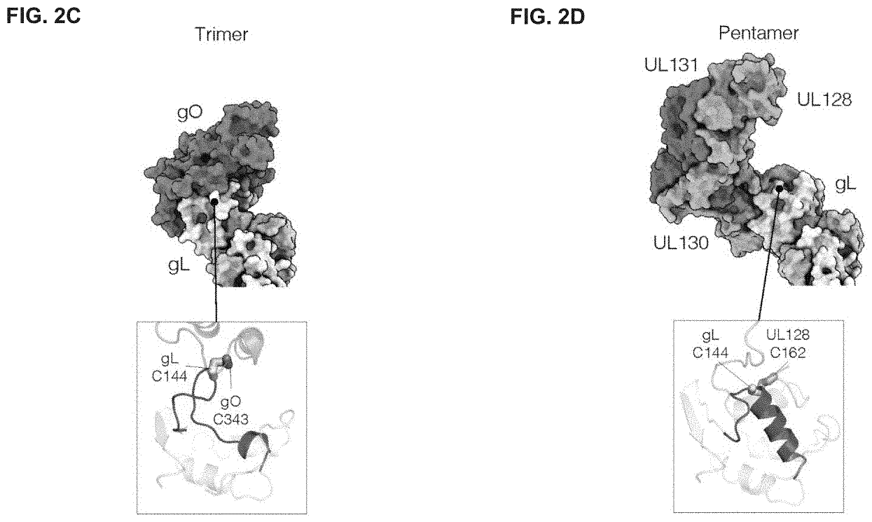

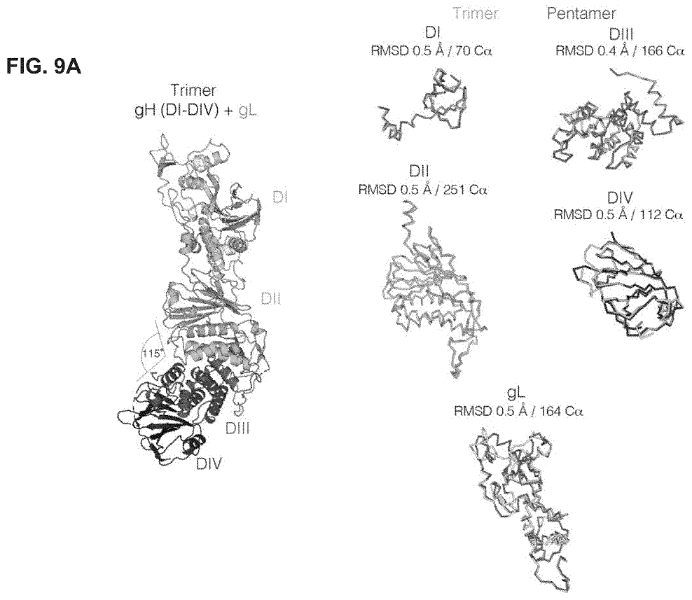

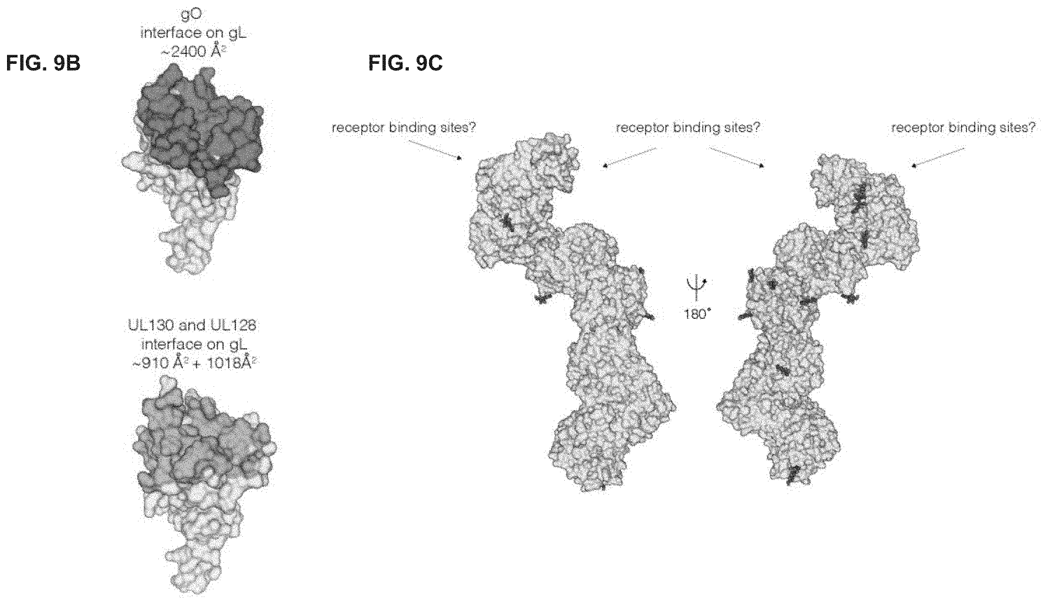

The structural determinants that mediate trimer and pentamer specific assemblies in HCMV have remained unknown. Both trimer and pentamer share their gH and gL subunits but differ in the composition of the distal subunits gO and UL128-131, respectively, which mediate receptor recognition (Ciferri et al., Proc Natl Acad Sci USA, 112: 1767-1772, 2015). In trimer, the four gH domains (DI-IV) extend linearly away from the membrane proximal face, where the N-terminal region of gH (DI) co-folds with gL near the membrane distal region of the molecule (FIGS. 1B and 9). Structural comparison of the gHgL subunits between the trimer and the crystal structure of the pentamer, bound to the UL130-specific neutralizing Fab 8121 (Chandramouli et al., Sci Immunol, 2: eaan1457, 2017), reveals almost identical structures for gH and gL in both complexes (RMSD 0.7 Å/582 Ca) (FIG. 2A). Accordingly, most glycosylated residues on gH and gL point towards the same directions for the trimer and pentamer with the exception of residue N641, which is found in a poorly resolved disordered loop region of the trimer structure (FIG. 2B).

Previously, gO and UL128/UL130/UL131A were established to bind to the same site on gHgL through formation of a disulfide bond with gL-Cys144 (Ciferri et al., Proc Natl Acad Sci USA, 112: 1767-1772, 2015), but the details for how the trimer and pentamer specific proteins can form a very stable interaction to the same gL interface have remained mysterious. An in-depth structural comparison of the individual gH domains and the gL subunit between trimer and pentamer confirms the expected high degree of structural similarity. Despite this overall similarity, we observed a key difference at the most distal end of the gL subunit, which interacts with either gO in the trimer or the UL128 and UL130 subunits in the pentamer (FIGS. 2C and 2D). gO binds as a capping crown around gL, covering ˜2400 Å² of its surface (FIG. 9B). The interaction surface between gL and gO spans a larger area than the corresponding interfaces between gL and either U1128 (≈1018 Å²) or UL130 (=910 Å²) in HCMV pentamer (FIG. 9B). A comparison between trimer and pentamer shows that, while the overall fold of gL is conserved, there are differences in the organization of the residues centered around the key Cys144 residue. Notably, in the trimer, this stretch of residues assumes a loop-like structure (FIG. 2C), whereas in the pentamer, this region folds into a regular alpha-helix to coordinate UL128 binding (FIG. 2D). Therefore, gL emerges as a key adaptor protein that has evolved to be able to recognize both gO or UL128/UL130 proteins through a structural switch centered around gL C144 to load the HCMV viral surface with trimer or pentamer complexes, depending on the cellular tropism and determining cellular tropism.

### Example 3. Binding Sites for HCMV Trimer and Pentamer Neutralizing Antibodies

An important goal of HCMV research is to understand the structural basis and mechanism of broadly neutralizing monoclonal antibodies (mAbs). Notably, mAbs isolated from healthy HCMV seropositive donors that target conformational-dependent epitopes of HCMV pentamer and trimer have been previously reported (Macagno et al., J Virol, 84: 1005-1013, 2010; Falk et al., J Infect Dis, 218: 876-885, 2018; Nokta et al., Antiviral Res, 24: 17-26, 1994). Among these, Msl-109 and 13H11 target gH in both HCMV trimer and pentamer complexes and are capable of broad HCMV neutralization (Nokta et al., Antiviral Res, 24: 17-26, 1994). Here, the new structure of the HCMV trimer gHgLgO-13H11-Msl-109 complex resolves the Fv regions of both Fabs and their corresponding epitopes on gH to high resolution (FIGS. 1A, 3A, and 8G).

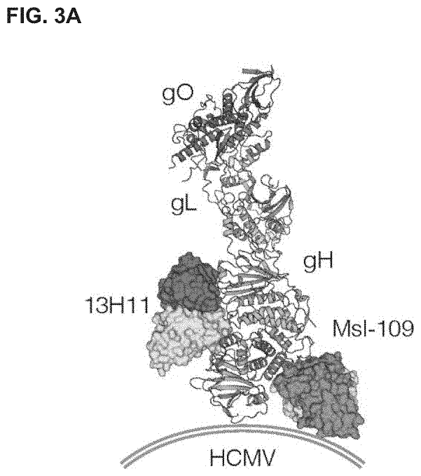

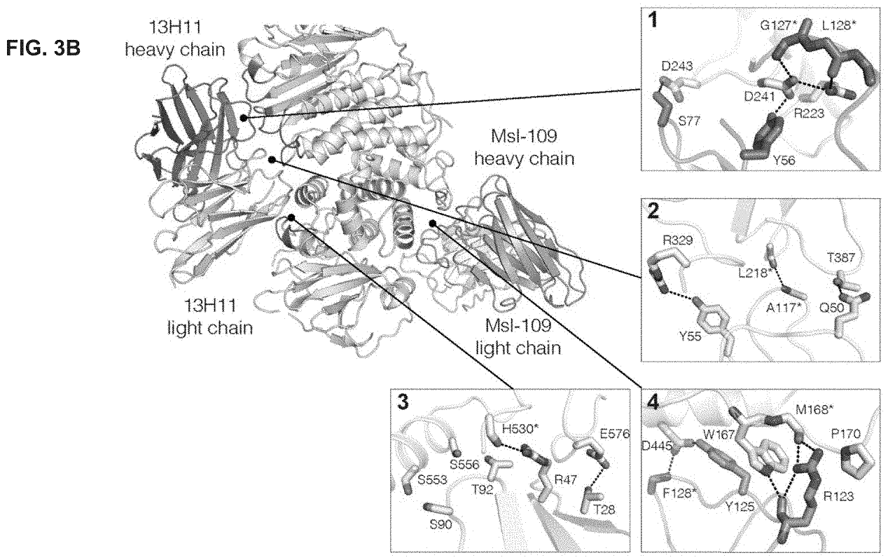

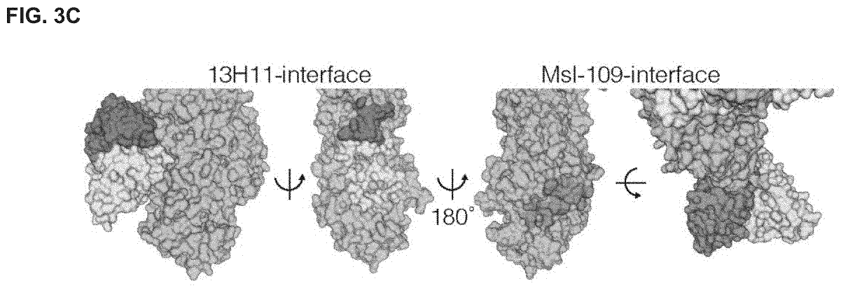

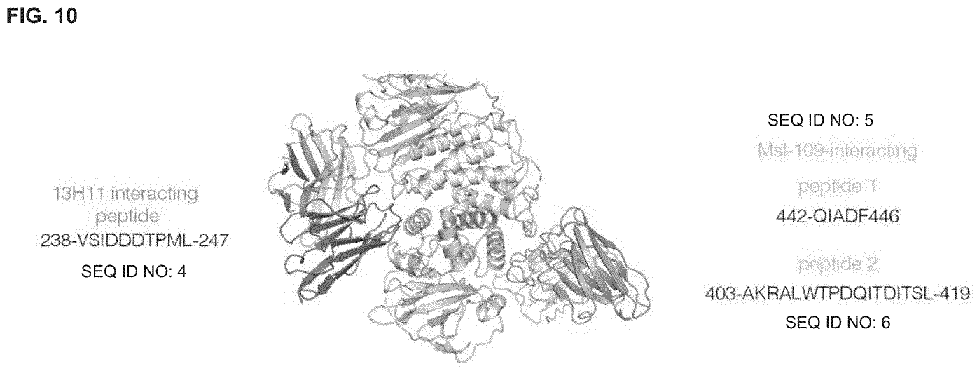

13H11 and Msl-109 bind on opposite faces of the kinked C-terminal region of gH. Using both heavy and light chains, 13H11 recognizes a large footprint of gH DII-DIII domains (FIGS. 3B and 3C). Specifically, 13H11 heavy chain engages residues R223, D241, D243 on gH DII domain through polar interactions (FIG. 3B, Panel 1). The light chain of 13H11 establishes polar contacts with the R329, L218 and T387 residues on gH-DII domain as well as residues S553, S556, H530 and E576 on gH-DIII domain (FIGS. 3B, Panel 2 and 3). In contrast to 13H11, Msl-109 utilizes its heavy chain to recognize the heel region of gH, recognizing a relatively small footprint of the DIII-DIV domains. The Msl-109 interactions involve polar contacts between its CDRs and residues W167, M168, P170 and D445 of gH-DIII and DIV domains. Notably, the residues contacted by Msl-109 are identical to the escape mutation positions (W167C/R, P170S/H, and D445N) that were isolated by growing HCMV VR1814 virus in epithelial cells or fibroblasts under suboptimal MSL-109 antibody concentrations (Fouts et al., Proc Natl Acad Sci USA, 111: 8209-8214, 2014). The newly established structures, including Fab contact regions on gH, significantly expand knowledge of the 13H11 and Msl-109 epitopes previously characterized by mass spectrometry methods (Ciferri et al., PLoS Pathog, 11: e1005230, 2015) (FIG. 10).

### Example 4. The Structure of gO Reveals a Novel Fold

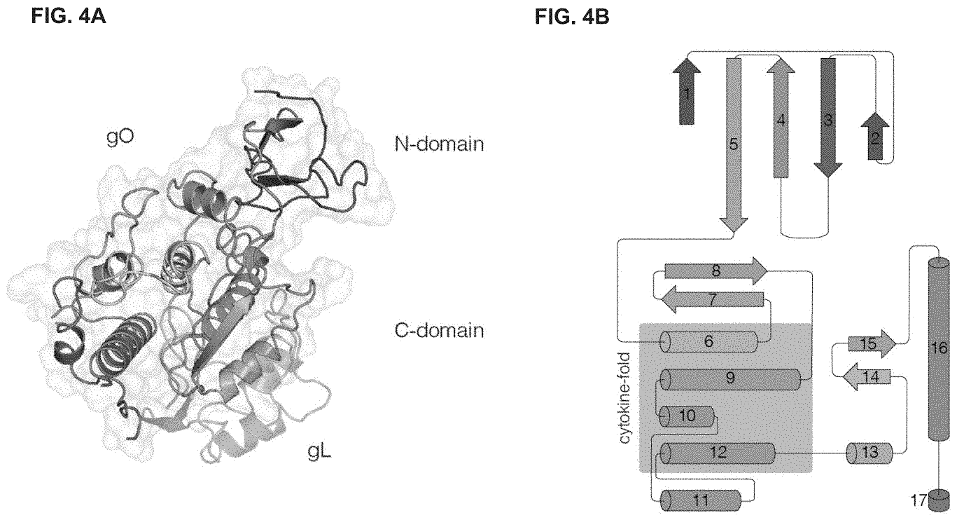

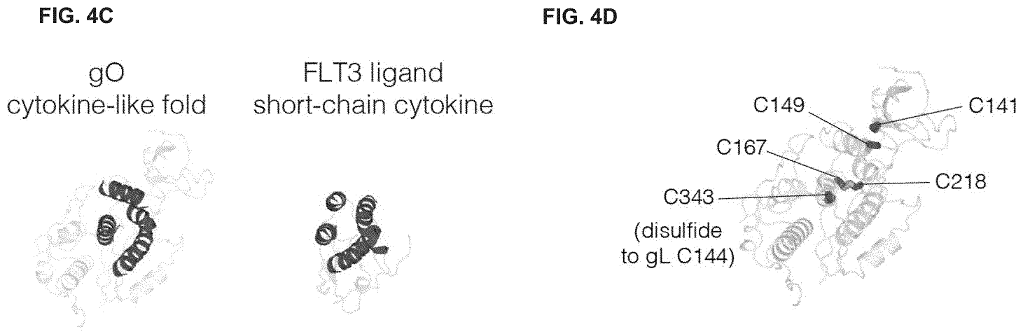

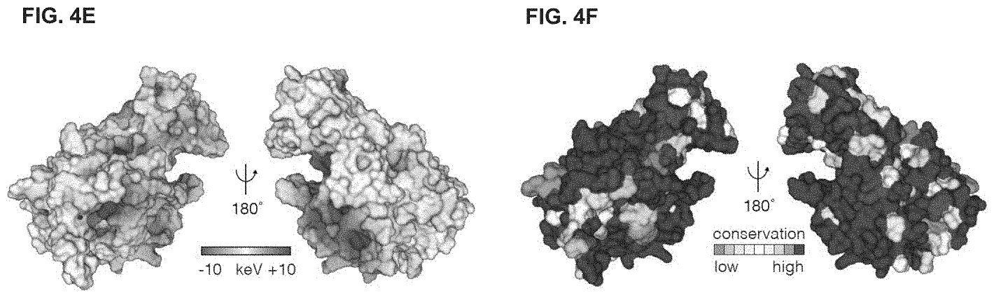

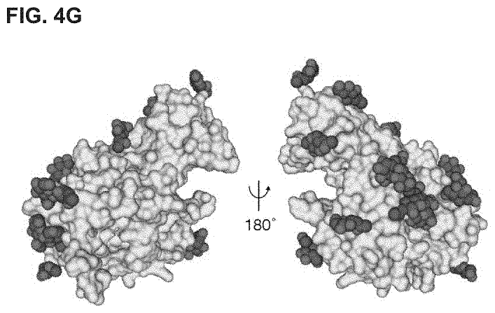

The structure of gO represents one of the most enigmatic HCMV glycoproteins as its amino-acid sequence does not align well to any previously published structures. In the cryo-EM structure described in Example 1, gO adopts a claw-like shape that is comprised of one N- and one C-terminal domain (FIG. 4A). The N-terminal globular domain consists of five beta strands, while the C-terminal domain is mainly alpha-helical. Remarkably, the central four alpha helices of the C-terminal domain share high structural similarities to a classical cytokine fold, with the closest member being FLT3 (FIGS. 4A-4C).

The two domains of gO are held together through two disulfides bridges mediated by Cys167-Cys₂₁₈ and Cys₁₄₉-Cys₁₄₁ (FIG. 4D) and align in one diagonal plane across the gO subunit including the disulfide between Cys₃₄₃(gO)-Cys₁₆₇(gL). All cysteines in gO are conserved across all HCMV strains, suggesting that this organization could be important for the function of the HCMV trimer and likely for receptor recognition. A comprehensive bioinformatic sequence analysis of the primary sequences of HCMV subunits indicated that gO is one of the least conserved envelope glycoproteins with a degree of conservation equal to 81% (Foglierini et al., Front Microbiol, 10: 1005, 2019). Notably, mapping of the gO conservation onto the newly established structure indicates that, while the overall conservation of gO is lower than other HCMV proteins, there are in fact large surface patches on both domains of gO that are highly conserved (FIG. 4E). Electrostatic surface analyses of gO identified a large area, comprising both N- and C-terminal domains, which is enriched in positive charges (FIG. 4F). This area overlaps with the conserved gO surface (FIGS. 4E-4F). Remarkably, the glycosylation sites on the gO subunit are unevenly distributed and clustered exclusively on one surface of the trimer, leaving one face of gO completely unmodified (FIG. 4G). This conserved, charged and glycosylation-free surface of gO was predicted to be the primary region involved in receptor binding.

### Example 5. Trimer Establishes Multiple Contacts with PDGFRα

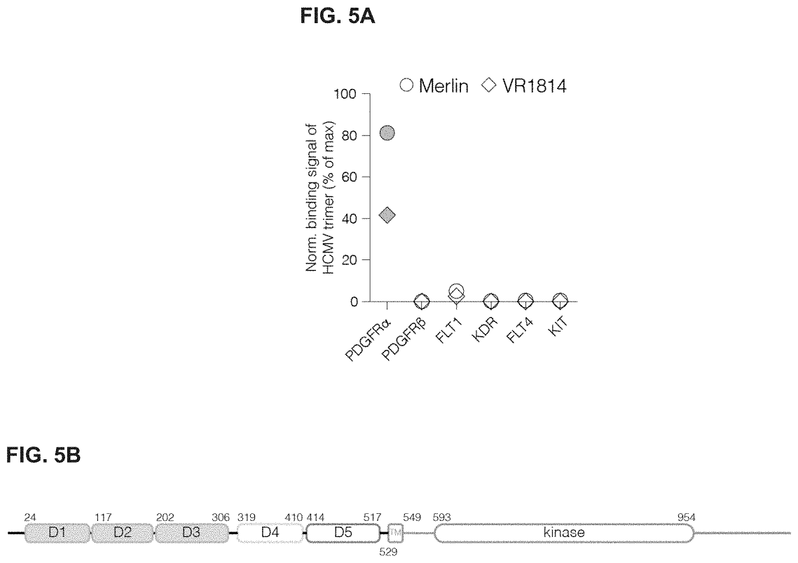

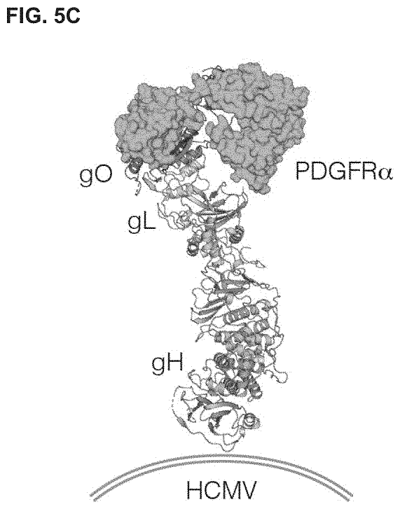

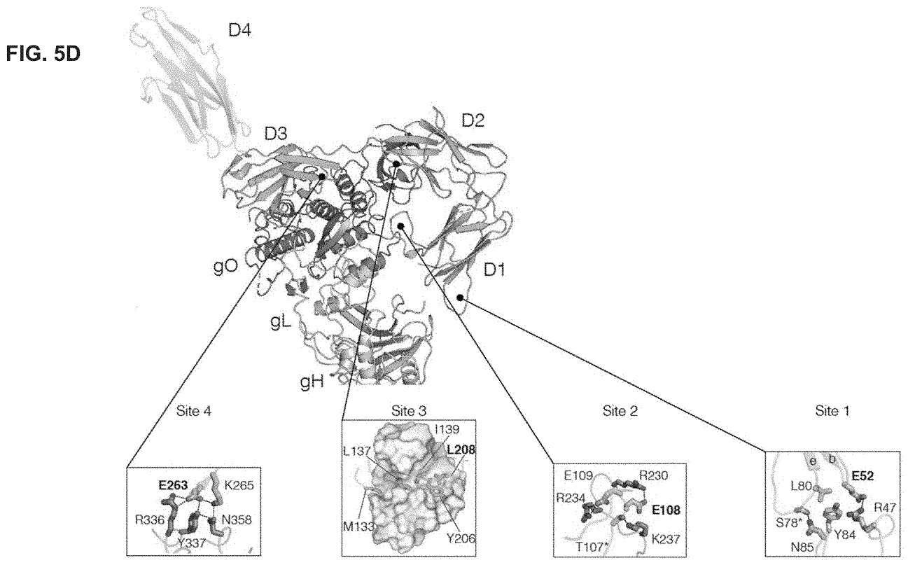

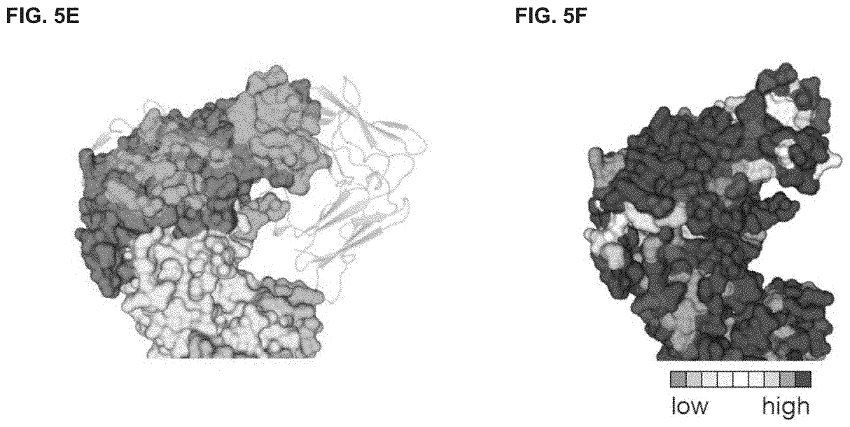

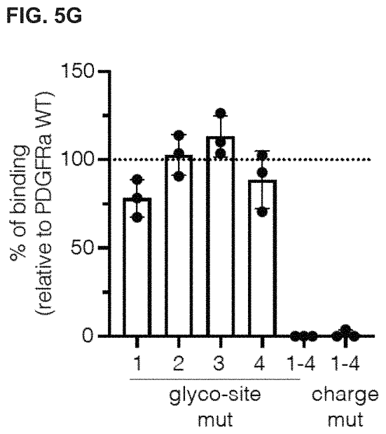

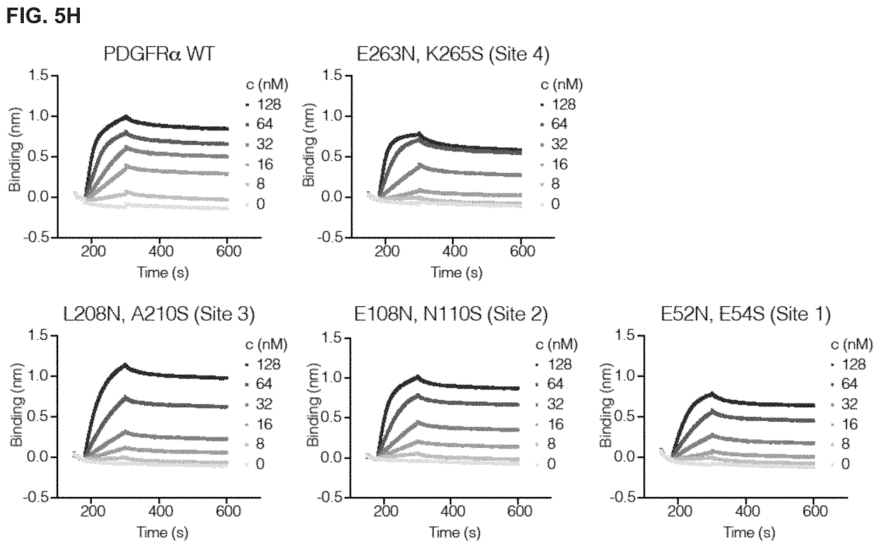

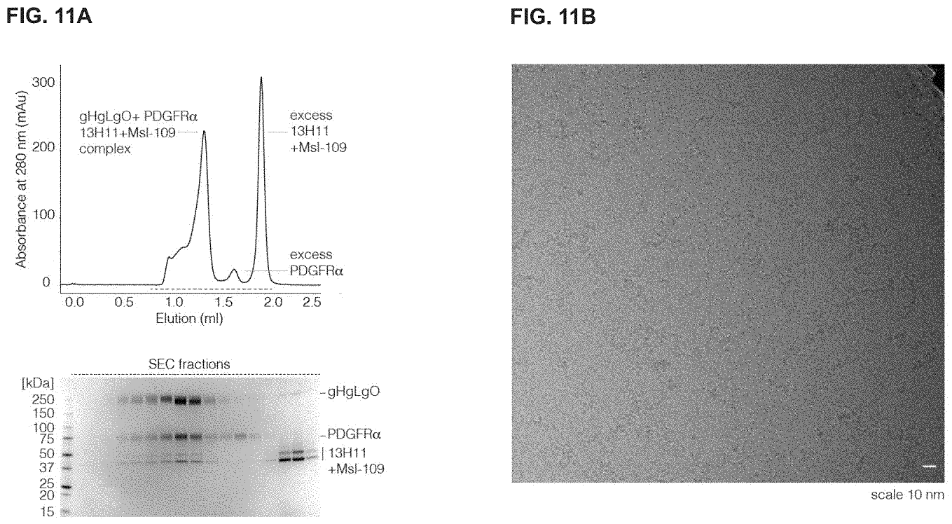

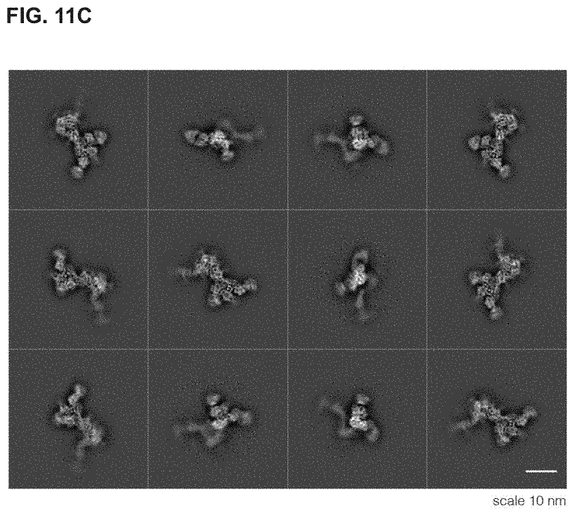

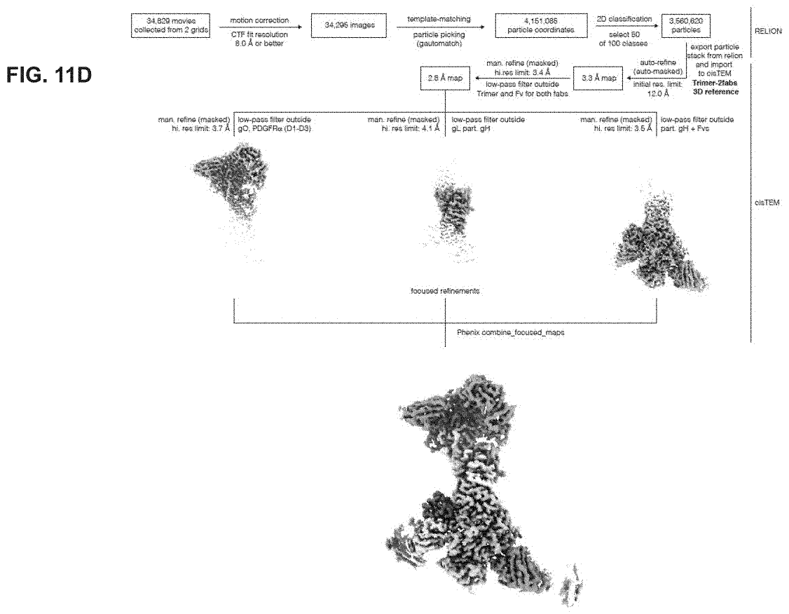

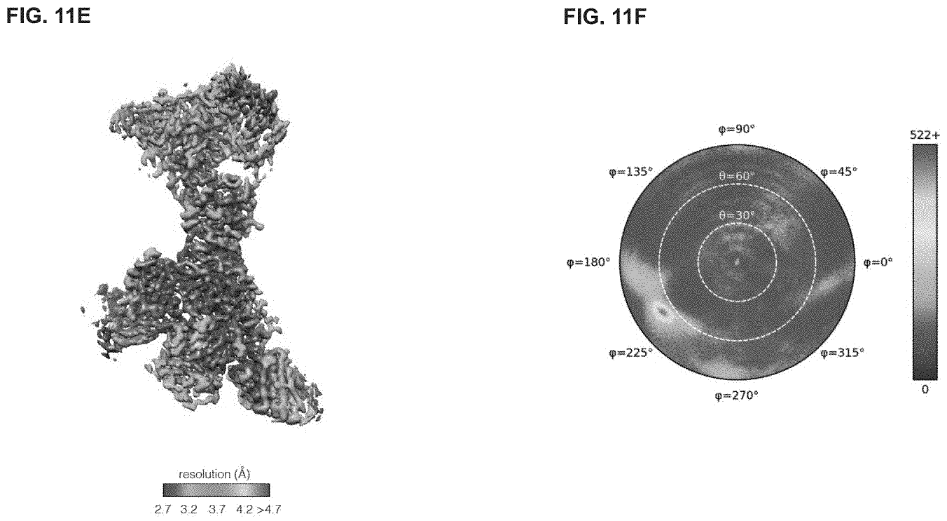

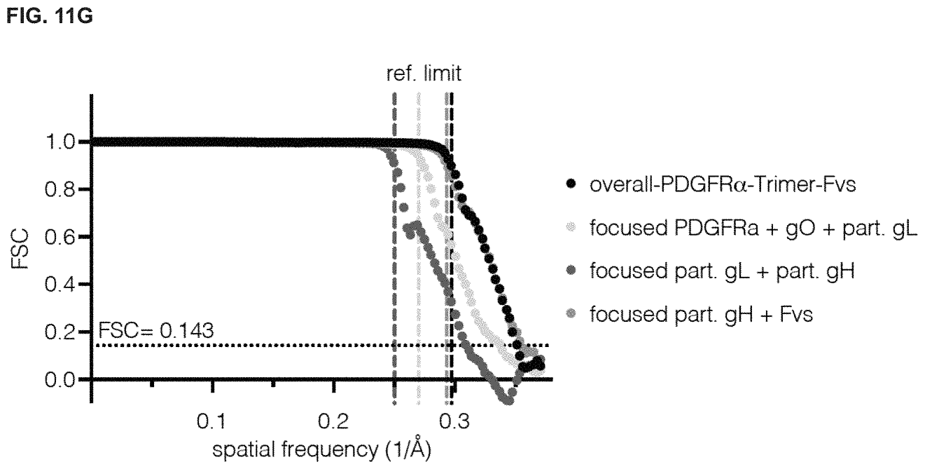

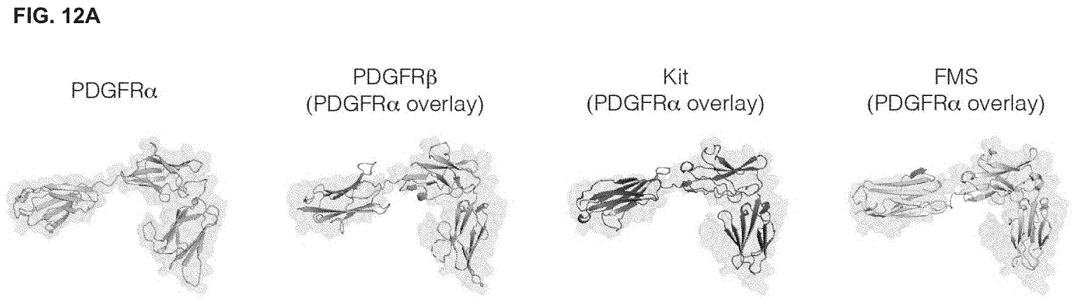

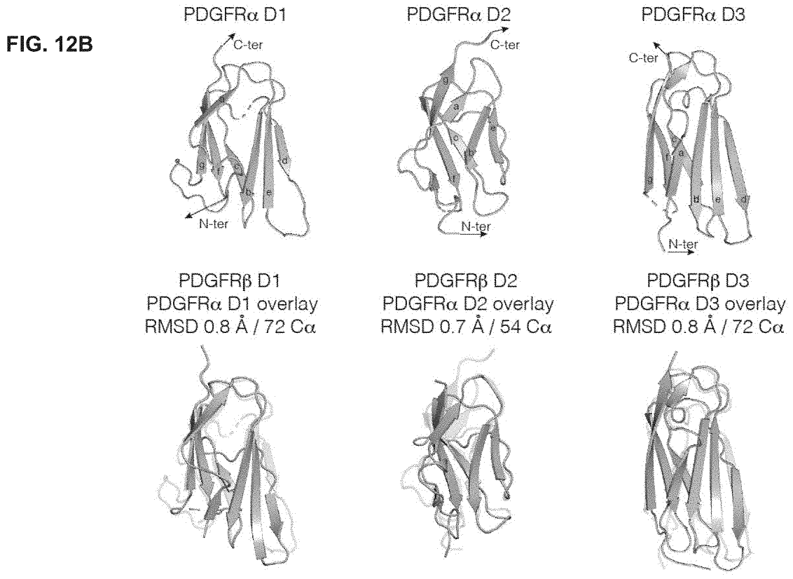

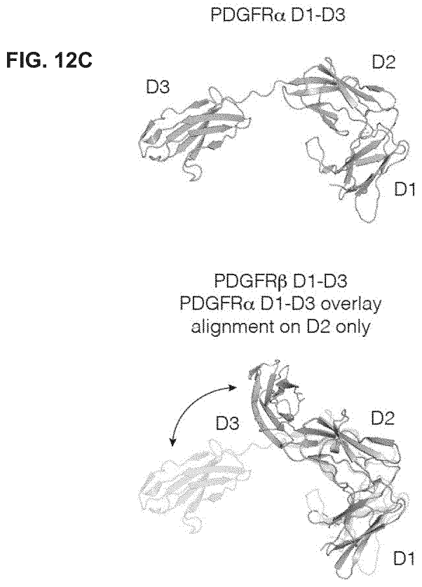

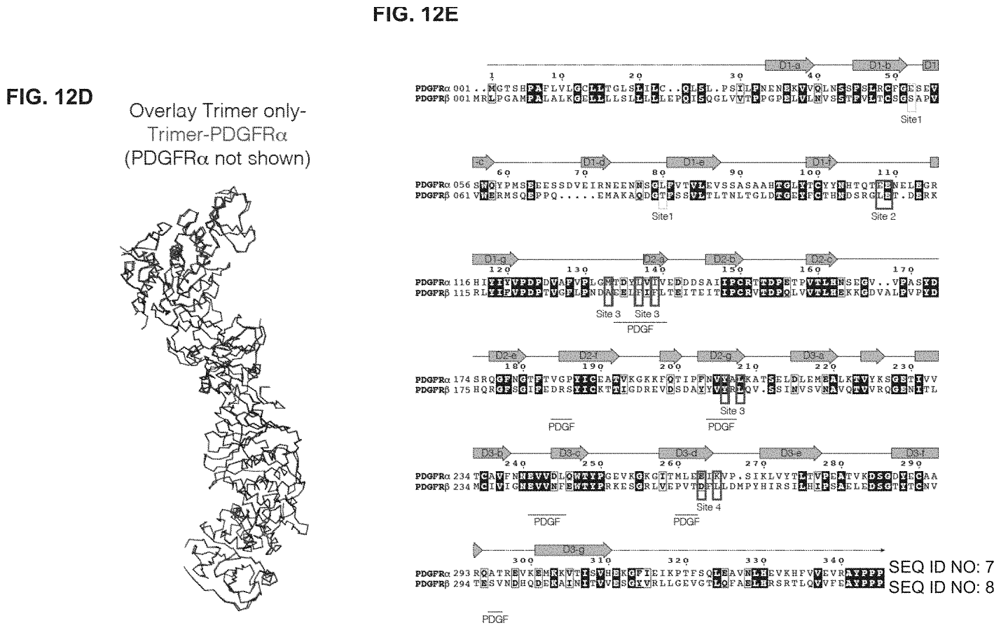

PDGFRα has recently been identified as a receptor for the HCMV trimer that is required for viral entry into fibroblasts (Martinez-Martin et al., Cell, 174: 1158-1171 e19, 2018; Kabanova et al., Nat Microbiol, 1: 16082, 2016; Wu et al., PLoS Pathog, 13: e1006281, 2017; Wu et al., Proc Natl Acad Sci USA, 115: E9889-E9898, 2018), but the structural basis for PDGFRα recognition has remained unknown. Notably, the HCMV trimer binds with high affinity and high selectivity to PDGFRα, as it does not bind to the closely related PDGFRβ or other members of class III receptor tyrosine kinases (RTKs) or the related VEGF receptors (FLT1, KDR, FLT4) (FIG. 5A). These interaction data with selected human receptor proteins were from the previously published Cell-Surface Receptor Discovery Platform results (FIG. 5A) (Martinez-Martin et al., Cell, 174: 1158-1171 e19, 2018). Members of the class III RTKs include PDGFRα, PDGFRβ, KIT, FMS, and FLT3, where the architecture of these receptors consists of five extracellular Ig-domain segments (D1-D5 domains), a short single span transmembrane domain, and an intracellular kinase domain (FIG. 5B). HCMV trimer was reconstituted in complex with Fabs 13H11/Msl-109 and the PDGFRα D1-D5 extracellular region and used cryoEM to determine its structure (FIG. 11A) to an overall resolution of ≈2.8 Å (FIGS. 5C and 11A-11G). The high-resolution of the cryo-EM map allowed for building the majority of the HCMV trimer-13H11-Msl109 complex and the amino acid sequence of the PDGFRα D1-D3 domains (FIGS. 11E-11F and 5C-5D). Density for the PDGFRα D4 and D5 domain was very weak, presumably due to the absence of direct contacts to the HCMV trimer. Possibly, the PDGFRα receptor interaction may propagate a conformational change onto the trimer to enable or disable binding of other HCMV glycoproteins, such as prefusion gB. However, almost identical conformations of trimer were observed when comparing structures before or after PDGFRα binding (FIG. 12D), suggesting a different mechanism is likely responsible for the activation of the HCMV fusion machinery.

When bound to trimer, PDGFRα D1-D3 adopted a kinked conformation similar to previously determined structures of PDGFRβ and other D1-D3 domains of class III RTKs (FIG. 12A). Structural comparisons of the individual D1-D3 domains between PDGFRα and PDGFRβ (determined in complex with PDGF) confirms the high similarity between both receptors (RMSDs between 0.7-0.8 Å, FIG. 12B). However, a comparison of the PDGFRα and PDGFRβ D1-D3 domains aligned along the D2 domain, showed a ˜105° relative rotation of D3 domain between the two structures, while the position of D1 was largely unchanged (FIG. 12C).

PDGFRα D1-D3 domains established extensive interactions at four major conserved surfaces across gO and the N terminus of gH (Site 1-4; FIGS. 5D-5F and Table 3). Specifically, the first major interaction surface (Site 1) involved the N-terminus of gH and loop regions of PDGFRα D1 between strands D1-b and D1-c and between strands D1-d and D1-e (FIGS. 5D and 12E). At Site 1, PDGFRα E52 formed a salt-bridge with gH R47 and PDGFRα S78 and L80 contact residues gH N85 and Y84, respectively (FIGS. 5D and 12E). Site 2 had an electrostatic nature with two acidic side chains in an extended loop between PDGFRα D1-f and D1-g (E108 and E109) that bound to the basic groove between the N- and C-terminal domains of gO (FIGS. 5D and 12E). At Site 3, hydrophobic residues at the PDGFRα D2 domain (M133, L137, I139, L208, Y206) oriented towards a hydrophobic groove at the N-terminal region of gO (FIG. 5D). Site 4 involved E263 and K265 on strand d at the PDGFRα D3 domain to establish charged and polar interactions with R336, Y337 and N358 on gO (FIG. 5D, Site 4). Notably, subtle but distinctive sidechain differences at each of these four major interaction sites may collectively rationalize the high specificity of the trimer to bind to PDGFRα but not to PDGFRβ (FIG. 12E). PDGFRα bound with low nanomolar affinity to the trimer (Table 2 and FIGS. 5G-5H), consistent with the four large contact sites observed in the structure. Interestingly, the introduction of mutations intended to add a bulky N-linked glycan to each individual site in gH or gO did not significantly reduce the binding of PDGFRα to the trimer (Table 2 and FIGS. 5G-5H). However, a combination of the N-glycan introducing mutations or the introduction of charge mutations at all four sites almost completely abolished the interaction between PDGFRα and the trimer (FIG. 5G). Thus, the extensive interaction interface between the trimer and PDGFRα consisted of numerous, highly conserved residues that facilitated a high affinity and highly selective binding of the trimer to the surface of the fibroblasts through engagement of PDGFRα. Residues involved in the binding of the HCMV gHgLgO trimer to PDGFRα are shown in Table 3.

TABLE 2
Binding affinities of HCMV gHgLgO trimer to PDGFRα-Fc wild type
or single-site point mutations.

| PDGFRα | K_{D} (M) |
|---|---|
| WT | 2.25 × 10⁻⁹ ± 1.1 |
| Site 1 | 1.65 × 10⁻⁸ ± 0.1 |
| Site 2 | 2.70 × 10⁻⁹ ± 2.1 |
| Site 3 | 2.15 × 10⁻⁸ ± 0.1 |
| Site 4 | 4.65 × 10⁻⁹ ± 3.1 |

Single site point mutations (Site 1: E52N, E54S; Site 2: E108N, N110S; Site 3: E208N, S210S; Site 4: E263N, K265S). K_{D}, dissociation constant; WT, wild type.

TABLE 3
Residues involved in the binding of HCMV gHgLgO trimer to PDGFRα

| Binding site | Trimer | PDGFRα |
|---|---|---|
| Site 1 | R47 of gH | E52 of PDGFRα |
| | Y84 of gH | S78 of PDGFRα |
| | N85 of gH | L80 of PDGFRα |
| Site 2 | R230 of gO | N103 of PDGFRα |
| | R234 of gO | Q106 of PDGFRα |
| | V235 of gO | T107 of PDGFRα |
| | K237 of gO | E108 of PDGFRα |
| | Y238 of gO | E109 of PDGFRα |
| Site 3 | N81 of gO | M133 of PDGFRα |
| | L82 of gO | L137 of PDGFRα |
| | M84 of gO | I139 of PDGFRα |
| | M86 of gO | E141 of PDGFRα |
| | F109 of gO | I147 of PDGFRα |
| | F111 of gO | S145 of PDGFRα |
| | T114 of gO | Y206 of PDGFRα |
| | Q115 of gO | L208 of PDGFRα |
| | R117 of gO | |
| | K121 of gO | |
| | V123 of gO | |
| Site 4 | R336 of gO | N240 of PDGFRα |
| | Y337 of gO | D244 of PDGFRα |
| | K344 of gO | Q246 of PDGFRα |
| | D346 of gO | T259 of PDGFRα |
| | N348 of gO | E263 of PDGFRα |
| | E354 of gO | K265 of PDGFRα |
| | N358 of gO | |

Protein Expression and Purification

Optimized coding DNA for human PDGFRα (1-528) was cloned into a pRK vector behind a CMV promoter. A C-terminal human IgG1 (Fc) tag was added to PDGFRα constructs. Expi293 cells in suspension were cultured in SMM 293T-I medium under 5% CO₂ at 37° C. and transfected using polyethylenimine (PEI) with DNAs at a 1:1:1 ratio for the gHgLgO expression when the cell density reached 4×10⁶ cells per ml. Transfected cells were cultured for 7 days before harvesting of the expression supernatant.

PDGFRα (1-524) with five amino acids at the C terminus (DDDDK) (Sino Biological) was used for cryo-EM sample preparation and the in vitro competition experiments. The lyophilized powder was resuspended in ddH2O, concentrated in an AMICON® Ultra centrifugal filter device (30 kDa MWCO) and purified on a Superose 6 3.2/300 column equilibrated in PDGFRα-SEC buffer (25 mM HEPES (pH 7.5), 250 mM NaCl) prior reconstitution with the HCMV trimer gHgLgO and the neutralizing Fabs.

Reconstitution of HCMV gHgLgO Trimer with PDGFRα and Neutralizing Fabs

The PDGFRα-gHgLgO-13H11-Msl-109 complex was assembled by incubation of 5 μM (83.3 μg) gHgLgO with an excess of PDGFRα at 6 μM (33.3 μg) and the Fabs 13H11 and Msl-109 at each 18 μM (50 μg) for at least 30 min on ice. The excess of the Fabs was removed by purification on a Superose 6 3.2/300 column equilibrated in SEC-reconst-2 buffer (25 mM HEPES (pH 7.5), 300 mM NaCl). The main peak fractions of gHgLgO-13H11-Msl-109 were combined and concentrated to 0.5 mg/ml for cryo-EM sample preparation.

Biolayer Interferometry

The interactions between the PDGFRα proteins and the CMV trimer were analyzed by biolayer interferometry using an Octet Red system. Recombinant PDGFRα proteins were captured onto anti human Fc-coated sensors (Forte Pall), and tested for binding to the CMV trimer as soluble analyte, assayed in PBS. Data was acquired using the Forte Pall software version 9.0. For comparison of relative binding between the WT trimer and the PDGFRα WT and mutant proteins, the trimer was assayed at 50 nM or 100 nM concentration and binding units at the end of the association were plotted. Low levels of PDGFRα proteins were captured on the sensors for estimation of binding kinetics. Data was acquired using the Octet Red instrument and subsequently the Biaevaluation software version 4.1 (GE Healthcare) was utilized for calculations of kinetic parameters.

Cryo-EM Sample Preparation and Data Acquisition

The PDGFRα-gHgLgO-13H11-Msl-109 complex was prepared as described in the following manner. Holey carbon grids (C-Flat 45 nm R 1.2/1.3 300 mesh coated with Au/Pd 80/20; Protochips) were glow-discharged for 10 s using the Solarus plasma cleaner (Gatan). The complex was gently cross-linked with 0.025% EM-grade glutaraldehyde for 10 min at room temperature and quenched with 9 mM Tris (pH 7.5). 3 μl of the sample (now at about 0.4 mg/ml) was applied to the grid. Grids were blotted with a Vitrobot Mark IV (Thermofisher) using 2.5-s blotting time with 100% humidity and plunge-frozen in liquid ethane cooled by liquid nitrogen.

Cryo-EM Data Processing

The PDGFRα-gHgLgO-13H11-Msl-109 complex was processed similarly as described in Example 1 for the gHgLgO-13H11-Msl-109 complex. A total of 34,829 movies were collected from two grids, corrected for frame motion using the MotionCor2 (Zheng et al. Nat Methods, 14(4): 331-332, 2017) implementation in RELION and contrast-transfer function parameters were fit using the 30-4.5 Å band of the spectrum with CTFFIND-4 (Rohou and Grigorieff, J Struct Biol., 192(2): 216-2, 2015). CTF fitted images were filtered on the basis of the detected fit resolution better than 8 Å. A total of 4,151,085 particles were picked by template-matching with gautomatch (MRC Laboratory of Molecular Biology) using a 30 Å low-pass filtered gHgLgO-13H11-Msl-109 complex reference structure. Particles were sorted during RELION 2D classification and 3,560,620 selected particles were imported into cisTEM for 3D refinements. The PDGFRα-gHgLgO-13H11-Msl-109 3D reconstruction was obtained after auto-refine and manual refinements with a mask, by applying low-pass filter (LPF) outside the mask (filter resolution 20 Å) and a score threshold of 0.25. The outside weight was thereby incrementally reduced from 0.5 to 0.15 in iterative rounds of manual refinements. The 3D reconstructions converged to a map resolution of 2.8 Å (Fourier shell correlation (FSC)=0.143, determined in cisTEM). To improve the quality of the map, focussed refinements were obtained after dividing the map into three distinct regions using masks and of the manual refinements low-pass filter (LPF) outside the mask as described above. The focussed maps were sharpened in cisTEM and combined using phenix as described above in Example 1.

Model Building and Structure Analysis

The structure of PDGFRβ (PDB: 3MJG) was used as a template for modelling of PDGFRα D1-D3. Model building and structure analysis was performed as in Example 1.

### Example 6. TGFβR3 Binds at the Interface Between gH, gL and gO

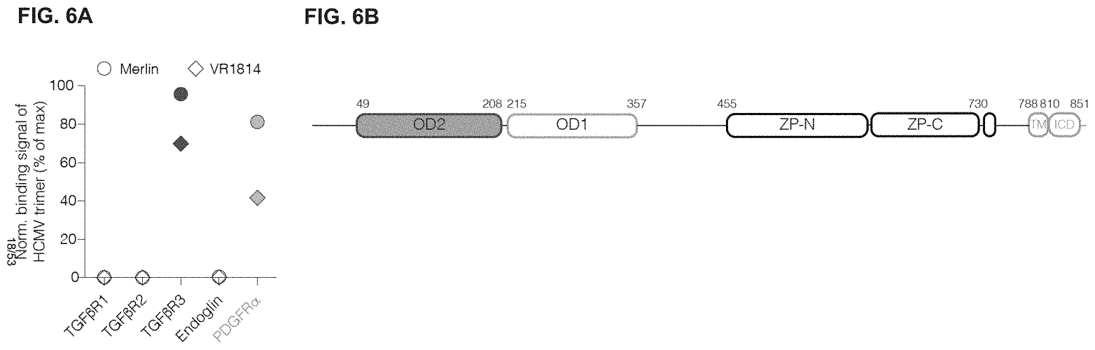

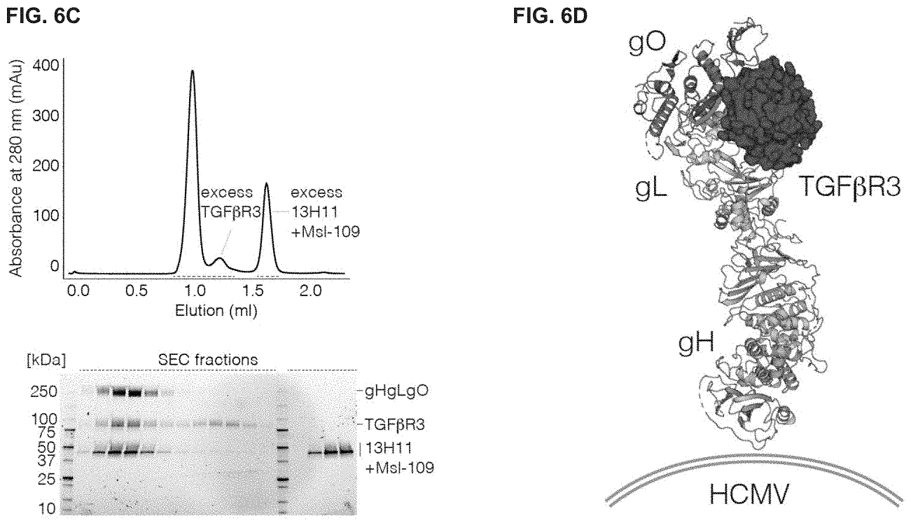

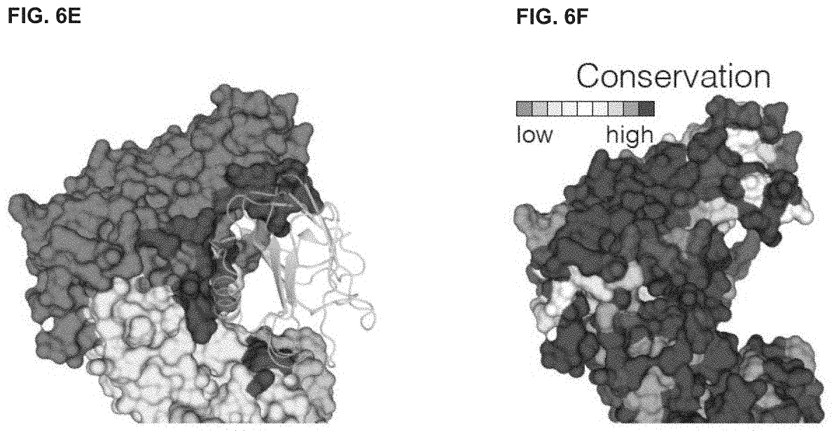

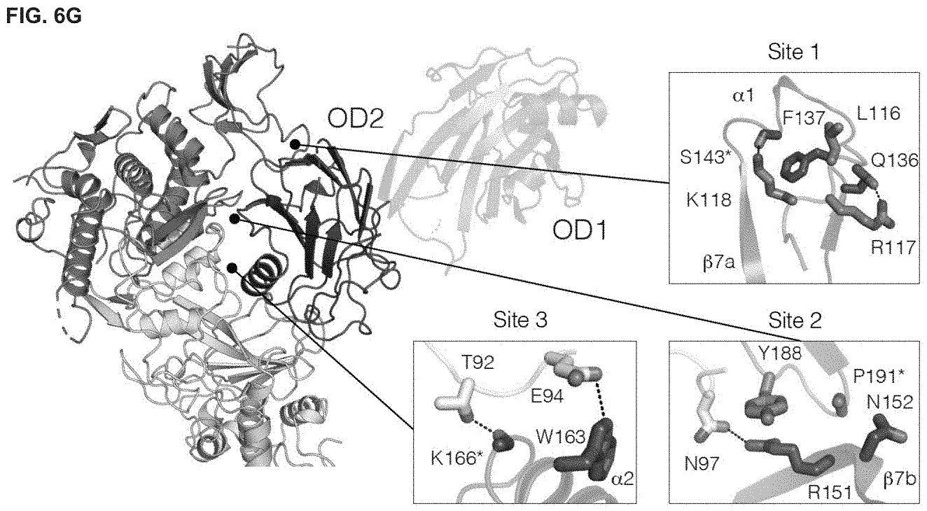

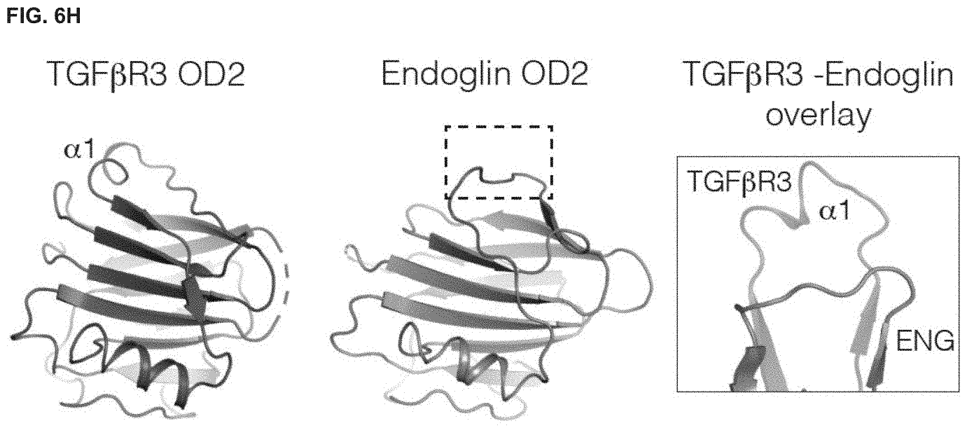

The trimer is required for HCMV tropism into all cell types, including endothelial and epithelial cells (Zhou et al., J Virol, 89: 8999-9009, 2015; Wille et al., mBio, 4: e00332-13, 2013; Ryckman et al., J Virol, 82: 60-70, 2008). This requirement suggests that the trimer may directly contribute to HCMV host-cell tropism by directly interacting with multiple receptors. Accordingly, TGFβR3 was recently identified as a high affinity binder to the trimer and a putative HCMV receptor (Martinez-Martin et al., Cell, 174: 1158-1171 e19, 2018). The TGFβR3 glycoprotein is a member of the TGF-beta signaling pathway receptor superfamily, which has essential roles in mediating cell proliferation, apoptosis, differentiation, and cellular migration in most human tissues (Zhang et al., Cold Spring Harb Perspect Biol, 9: a022145, 2017). The extracellular domain of TGFβR3 is composed of two N-terminal membrane-distal orphan domains (OD2 and OD1) and the membrane-proximal zona pellucida (ZP) domain (Kim et al., Structure, 27: 1427-1442 e4, 2019). Each OD is comprised of two p sandwich domains, while the ZP domain adopts a classical immunoglobulin-like fold (FIG. 6B) (Lin et al., Proc Natl Acad Sci USA, 108: 5232-5236, 2011). Despite the homology between TGFβR3 and TGFβR1, TGFβR2 or Endoglin, no binding between these additional proteins and HCMV trimer was observed (FIG. 6A) (Martinez-Martin et al., Cell, 174: 1158-1171 e19, 2018), which was previously published in the Cell-Surface Receptor Discovery Platform results.

TABLE 4
Residues involved in the binding of HCMV gHgLgO trimer to TGFBR3

| Binding site | Trimer | TGFβR3 |
|---|---|---|
| Site 1 | Q115 of gO | S143 |
| | L116 of gO | V135 of TGFβR3 |
| | R117 of gO | Q136 of TGFβR3 |
| | K118 of gO | F137 of TGFβR3 |
| | | S143 of TGFβR3 |
| Site 2 | Y188 of gO | R151 of TGFβR3 |
| | P191 of gO | N152 of TGFβR3 |
| | N97 of gL | E167 of TGFβR3 |
| Site 3 | E94 of gL | W163 of TGFβR3 |
| | T92 of gL | K166 of TGFβR3 |

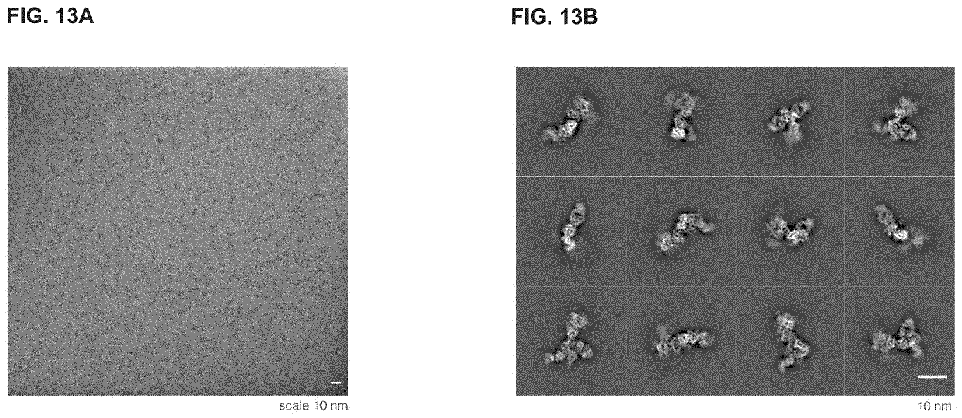

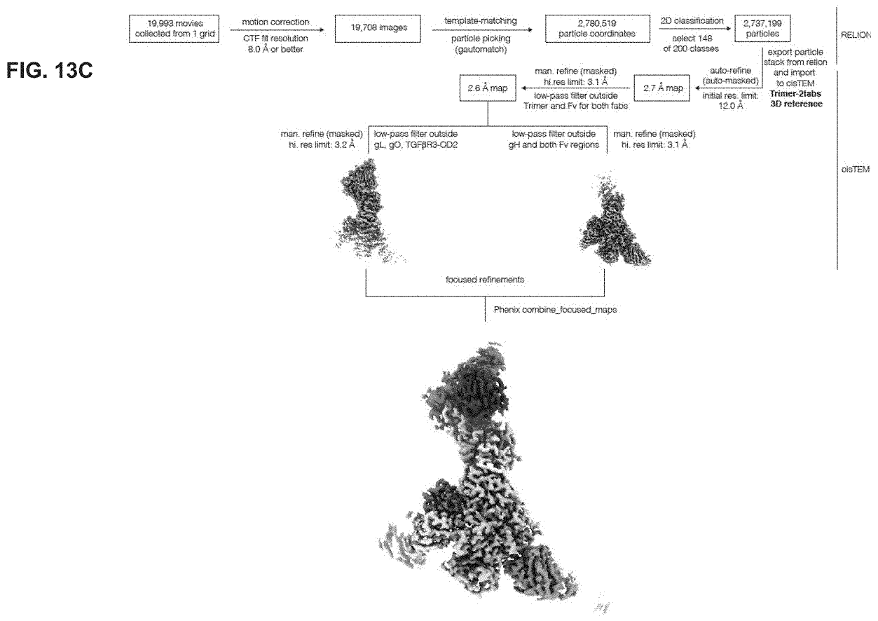

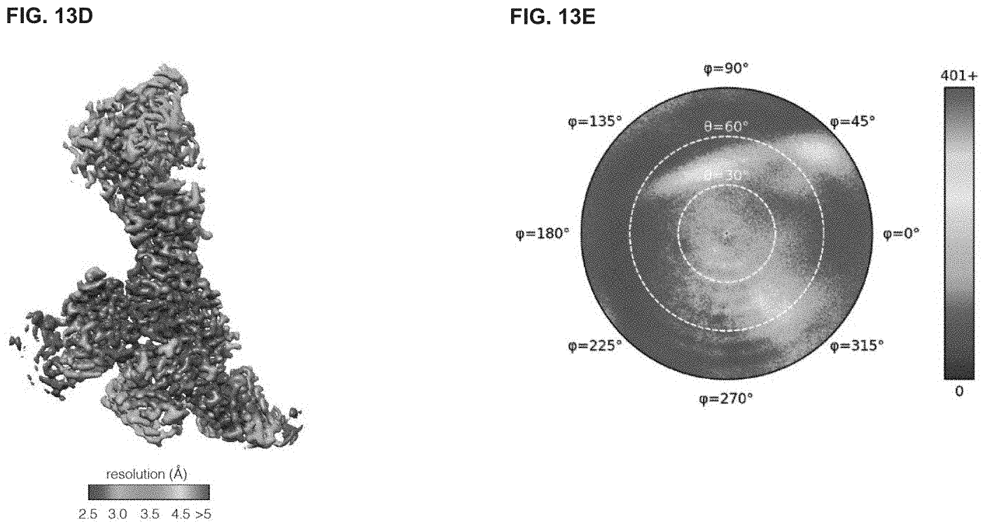

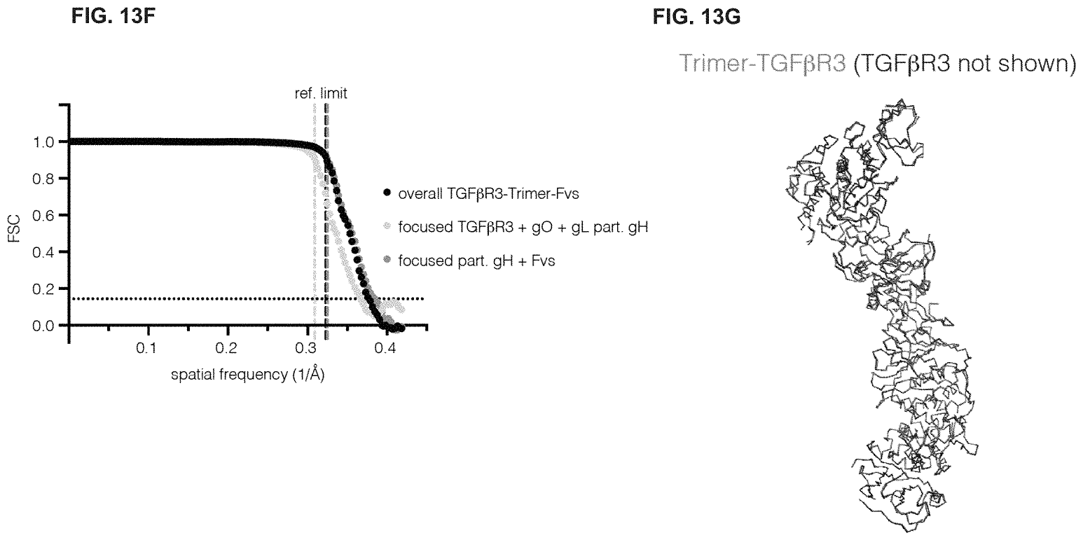

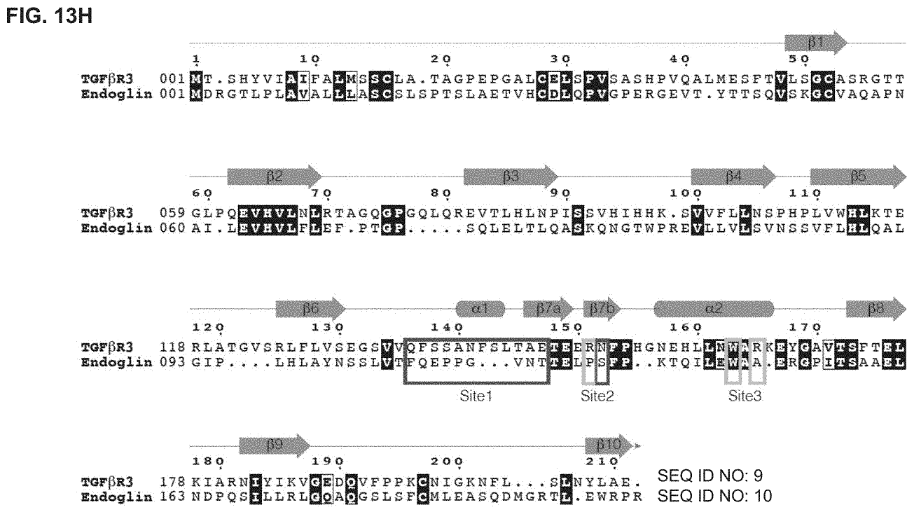

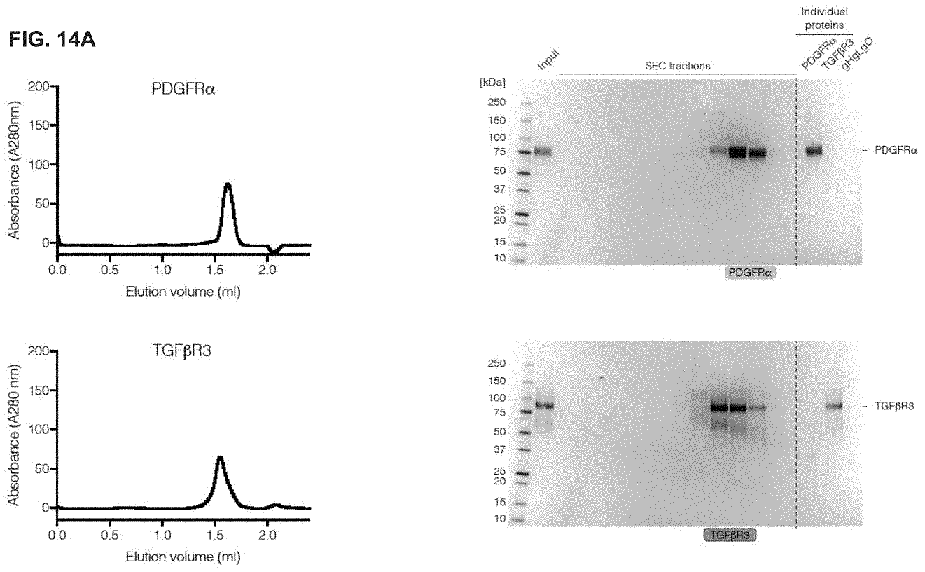

To gain direct structural insights into TGFβR3 recognition by trimer, a stoichiometric complex of TGFβR3 was reconstituted containing the OD and ZP domains with the HCMV trimer and the Fabs 13H11 and Msl-109 and cryo-EM was used to determine the structure to an overall resolution of ≈2.6 Å (FIGS. 6C-6G and 13A-13F). Because the trimer associated with the Ig-like D1-D3 domains of PDGFRα, the binding of TGFβR3 was anticipated to occur through its Ig-like domains. Unexpectedly, the newly revealed structure indicated that TGFβR3 exclusively utilized the OD2 domain to bind to conserved residues on gO and gL at three major sites (FIGS. 6D-6G and Table 4), whereas the density for the TGFβR3 OD1 domain appeared weak, apparently due to the absence of direct contacts to the trimer. TGFβR3 OD1 did not make specific contact to the HCMV trimer, was poorly resolved in the cryo-EM map and not modelled in the structure. Notably, binding of TGFβR3 did not induce any major structural rearrangement on the trimer (FIG. 13G), similarly to what was observed for PDGFRα.

Human TGFβR3 OD2 domain was comprised of 10 β strands and two α-helices, one between β6 and β7 (α1), and the other one between β7 and β8 (α2) (FIG. 13H), and the regions surrounding both helices made key contacts with the HCMV trimer. Specifically, TGFβR3 utilized a looped structure surrounding the α1 region to hydrogen bond with the S₁₄₃ carbonyl and Q₁₃₆ sidechain to the gO sidechains K₁₁₈ and R₁₁₇, respectively, at the N-terminal domain of gO (Site 1, FIGS. 6G and 13H). Hydrophobic contacts between TGFβR3 F₁₃₇ and gO L₁₁ further supported the interaction at Site 1 (FIG. 6G, Site 1). At Site 2, TGFβR3 R₁₅₁ at strand β7b formed a pi-stacking interaction with gO Y₁₈₈ and hydrogen bonds to gL N₉₇ (FIG. 6G, Site 2). At Site 3, TGFβR3 W₁₆₃ at α2 and the carbonyl of K₁₆₆ formed hydrogen bonds with the gL sidechains E₉₄ and T₉₂, respectively (FIG. 6G, Site 3).

The OD2 domains of TGFβR3 and endoglin were highly similar in structure (FIG. 6H). A detailed view, especially at the key interaction areas of Site 1 and 2, revealed that the endoglin OD2 domain lacks the looped structure at α1 and did not adopt a beta-strand secondary structure at β7b (FIGS. 13H and 6H). Overall, the multitude of interacting residues with contrasting properties rationalized the strong interaction between TGFβR3 and the HCMV trimer, and the key differences to endoglin at Site 1 and Site 2 provided an explanation for the high specificity and selectivity of the TGFβR3-trimer interaction.

Protein Expression and Purification

Optimized coding DNA for human TGFβR3 (1-787) was cloned into a pRK vector behind a CMV promoter. A C-terminal FLAG-tag was added to TGFβR3 constructs. Expi293 cells in suspension were cultured in SMM 293T-I medium under 5% CO₂ at 37° C. and transfected using polyethylenimine (PEI) with DNAs at a 1:1:1 ratio for the gHgLgO expression when the cell density reached 4×10⁶ cells per ml. Transfected cells were cultured for 7 days before harvesting of the expression supernatant.

Human TGFβR3-Flag was purified from a 101 expression supernatant. The supernatant was incubated with 10 ml M2 agarose Flag resin (Sigma) and incubated for 20 h at 4° C. The resin was washed with 10 CV FLAG-wash Buffer (30 mM HEPES (pH 7.5), 300 mM NaCl, 5% glycerol) and eluted with FLAG-wash buffer supplemented with 0.2 mg/ml FLAG peptide. The eluate was concentrated with an AMICON® Ultra Centrifugal filter device (30 kDa MWCO) and loaded on a Superdex 200 10/60 column equilibrated in TGFβR-SEC-1 buffer (30 mM HEPES (pH 7.5), 300 mM NaCl, 5% glycerol).

TGFβR3 (1-781) with a C-terminal HIS-tag (Sino Biological) was used for cryo-EM sample preparation. The lyophilized powder was resuspended in ddH2O, concentrated in an AMICON® Ultra Centrifugal filter device (30 kDa MWCO) and purified on a Superdex 200 3.2/300 column equilibrated in TGFβR-SEC-2 buffer (25 mM HEPES (pH 7.5), 200 mM NaCl) prior to assembling with the HCMV trimer gHgLgO and the neutralizing Fabs.

Reconstitution of HCMV gHgLgO Trimer with Human TGFβR3 and Neutralizing Fabs

The TGFβR3-gHgLgO-13H11-Msl-109 complex was assembled by incubation of 7.6 μM (85.5 μg) gHgLgO with an excess of TGFβR3 at 9.2 μM (54 μg) of the Fabs 13H11 at 22.6 μM (78 μg) and Msl-109 at 61 μM (210 μg) for at least 30 min on ice. The excess of the Fabs was removed by purification on a Superose 6 3.2/300 column equilibrated in SEC-reconst-2 buffer (25 mM HEPES (pH 7.5), 300 mM NaCl). The main peak fractions of gHgLgO-13H11-Msl-109 were combined and concentrated to 0.5 mg/ml for cryo-EM sample preparation.

Cryo-EM Sample Preparation and Data Acquisition

The TGFβR3-gHgLgO-13H11-Msl-109 complex was prepared in the following manner. Holey carbon grids, (Ultrafoil 25 nM Au R 1.2/1.3 300 mesh; Quantifoil) were glow-discharged for 10 s using the Solarus plasma cleaner (Gatan). 3 μl of the sample was applied to the grid and blotted single-sided with a Leica EM GP (Leica) using 3.5-s blotting time with 100% humidity and plunge-frozen in liquid ethane cooled by liquid nitrogen.

The TGFβR3-gHgLgO-13H11-Msl-109 complex was processed similarly as described above in Example 1 for the gHgLgO-13H11-Msl-109 complex. A total of 19,993 movies were corrected for frame motion using the MotionCor2 (Zheng et al. Nat Methods, 14(4): 331-332, 2017) implementation in RELION and contrast-transfer function parameters were fit using the 30-4.5 Å band of the spectrum with CTFFIND-4 (Rohou and Grigorieff, J Struct Biol., 192(2): 216-2, 2015). A total of 2,780,519 particles were picked by template-matching with gautomatch using a 30 Å low-pass filtered gHgLgO-13H11-Msl-109 complex reference structure. Particles were sorted during RELION 2D classification and 2,780,519 selected particles were imported into cisTEM for 3D refinements. The TGFβR3-gHgLgO-13H11-Msl-109 3D reconstruction was obtained after auto-refine and manual refinements with a mask and by applying low-pass filter (LPF) outside the mask (filter resolution 20 Å) and a score threshold of 0.25. The outside weight was thereby incrementally reduced from 0.5 to 0.15 in iterative rounds of manual refinements. The 3D reconstructions converged to a map resolution of 2.6 Å (Fourier shell correlation (FSC)=0.143, determined in cisTEM). To improve the quality of the map, focussed refinements were obtained after dividing the map into two distinct regions using masks and of the manual refinements low-pass filter (LPF) outside the mask as described above. The focussed maps were sharpened in cisTEM and combined using phenix as described above. Local resolution was determined in cisTEM using an in-house re-implementation of the blocres algorithm (Cardone et al 2013).

Model Building and Structure Analysis

The structure of zebrafish TGFβR3 (PDB: 6MZN) was used as a template for modelling human TGFβR3 OD2. Model building and structure analysis was performed as in Example 1.

### Example 7. PDGFRα and TGFβR3 Compete for HCMV Trimer Binding

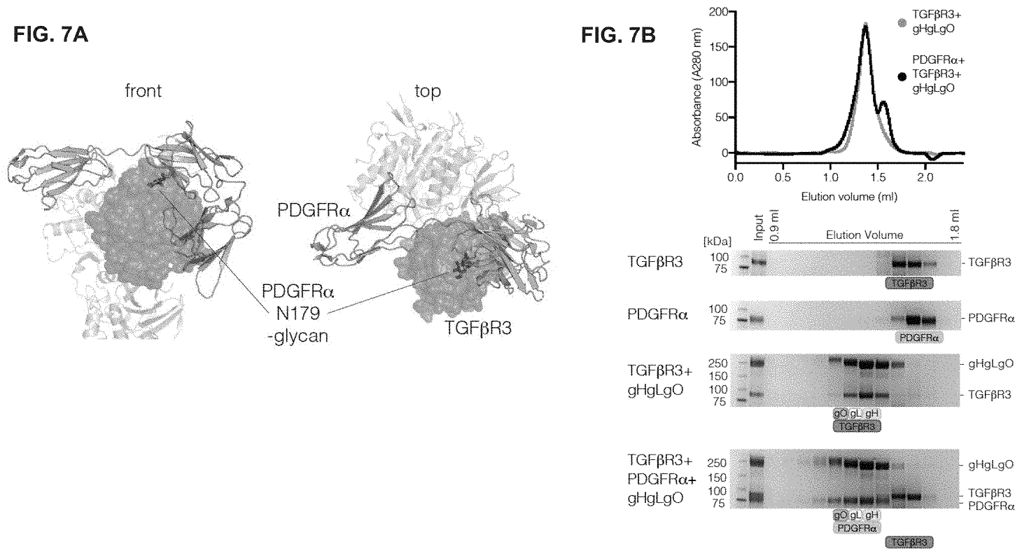

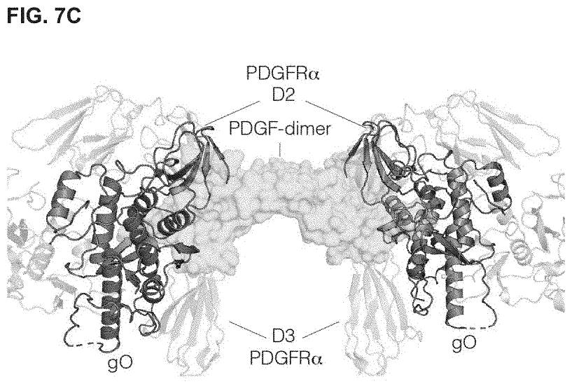

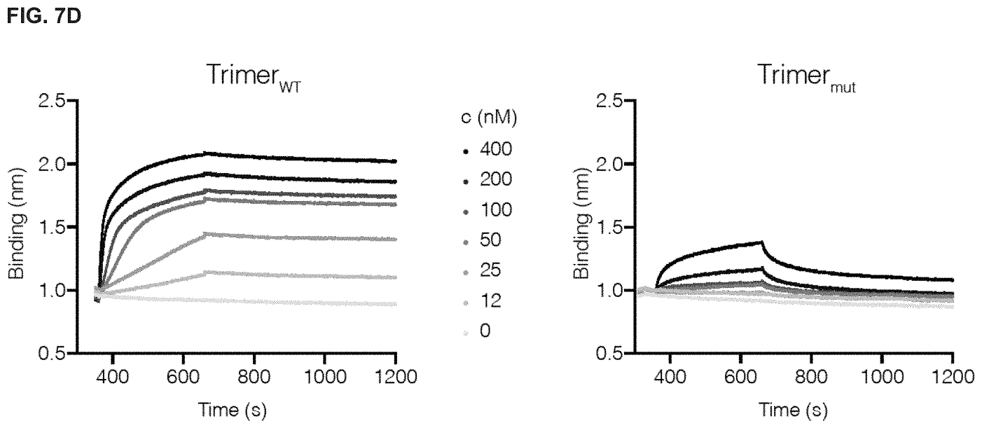

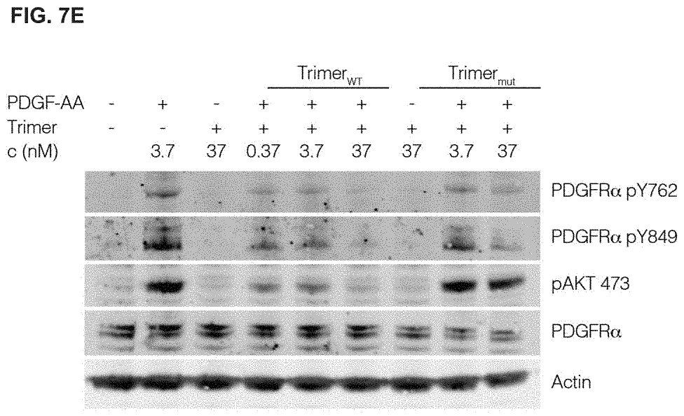

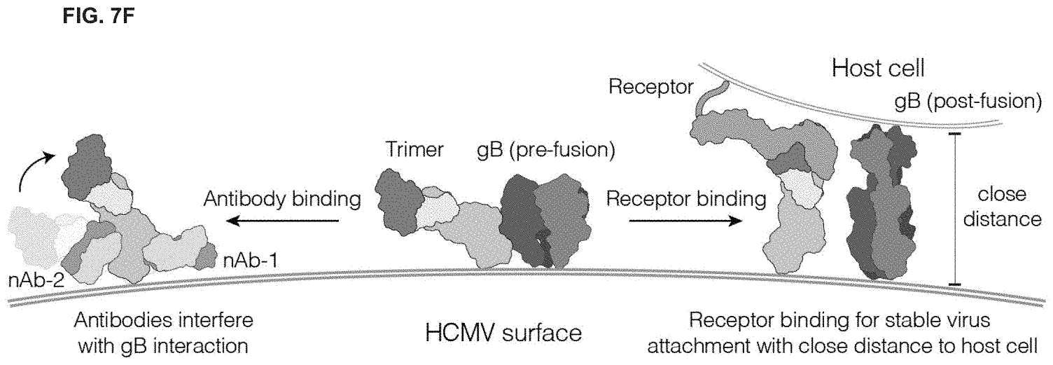

The HCMV trimer was able to bind with high affinity to two completely different domain architectures present on divergent receptors: the Ig-like D1-D3 domains of PDGFRα and the OD2 domain of TGFβR3 (FIGS. 5 and 6). While both receptors interacted at the membrane distal region of the trimer, PDGFRα and TGFβR3 bound to the trimer across different interaction surfaces (FIGS. 5E and 6E). Despite this, superposition of the trimer-PDGFRα and trimer-TGFβR3 complex structures suggested that these receptors share a partially overlapping binding site at the interface between gH, gL and gO and therefore cannot bind the trimer at the same time (FIG. 7A). Moreover, an N-linked glycan chain on PDGFRα N₁₇₉ was found to point in the direction of the TGFβR3 binding site, which might further limit simultaneous receptor binding (FIG. 7A).

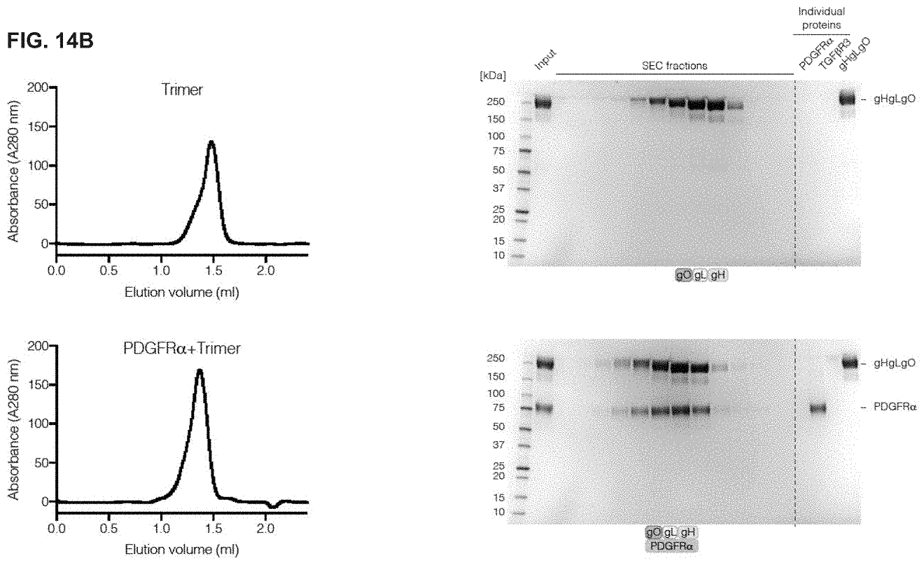

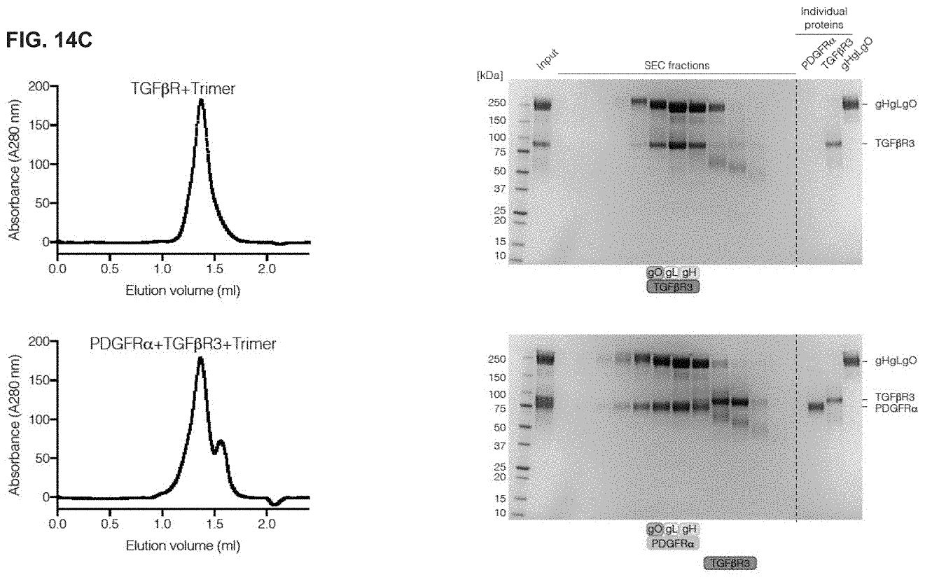

To test the hypothesis that PDGFRα and TGFβR3 binding to trimer are mutually exclusive, competition experiments were performed by incubating HCMV trimer bound to TGFβR3 with equimolar amounts of PDGFRα (FIGS. 7B and 14). The results indicated that PDGFRα can completely substitute for bound TGFβR3 (FIG. 7B), consistent with the reported higher affinity between HCMV trimer and PDGFRα as compared to TGFβR3 (Martinez-Martin et al., Cell, 174: 1158-1171 e19, 2018). Taken together, these structural and biophysical data suggest that PDGFRα and TGFβR3 do not function as co-receptors, but rather are more likely to mediate HCMV tropism acting as independent receptors.

Binding Competition Experiment of PDGFRα and TGFβR3 to HCMV Trimer gHgLgO

HCMV trimer gHgLgO, PDGFRα and TGFβR3 alone or a combination of gHgLgO+PDGFRα, gHgLgO+TGFβR3 or gHgLgO+PDGFRα+TGFβR3 were co-incubated at a concentration of 3 μM in SEC-competition buffer (25 mM HEPES (pH 7.5), 300 mM NaCl) for at least 60 min on ice and loaded on a Superose 6 3.2/300 column equilibrated in SEC-competition buffer.

### Example 8. The HCMV Trimer Competes with the Growth Factor PDGF for Binding to PDGFRα

In Examples 4-6, cryo-EM structures of the trimer, trimer-PDGFRα and trimer-TGFβR3 revealed the functionally important and highly conserved surfaces on the trimer involved in receptor binding and the likely target of potent neutralizing antibodies (FIGS. 4, 5, and 6). Thus, it was investigated whether the trimer interactions with PDGFRα might interfere with key cellular signaling pathways. For PDGFRα, binding of the PDGF growth factors dimerizes the receptor and activates the intracellular kinase domain to induce the signaling cascade (Shim et al., Proc Natl Acad Sci USA, 107: 11307-11312, 2010). A structural superposition of the trimer-PDGFRα complex with a homology model of the dimeric (signaling active) PDGFRα-PDGF complex showed multiple steric clashes of gO and PDGF at the PDGFRα D2 and D3 interaction interfaces (FIGS. 7C and 12E). Given the strong interaction of the trimer and PDGFRα at low nanomolar affinities (Kabanova et al., Nat Microbiol, 1: 16082, 2016) (Table 2) and the moderate binding affinity of PDGF-AA to PDGFRα characterized by three digit nanomolar affinities (Mamer et al., Sci Rep, 7: 16439, 2017), the hypothesis that the trimer can compete with PDGF for binding to PDGFRα and thereby prevent the induction of the signaling cascade was tested. To test this hypothesis, the HCMV trimer was first purified with charge mutations at gO (Trimerₘᵤₜ; Site 2-4: M84R, F111R, R117E, F136R, R212E, R230E, R234E, R336E, F342E, A351R, N358R) and an approximately 10,000 times reduced in vitro binding to PDGFRα was observed (KD_{Trimer-WT}: 2.25×10⁻⁹+/−1.1 M vs. KD_{Trimer-mut}: 4.25×10⁻⁵+/−0.5 M) (FIG. 7D). Next, PDGFRα activation and signaling in MRC-5 fibroblast cells was assessed upon addition of PDGF-AA and Trimer_{WT} or Trimerₘᵤₜ by detecting the auto-phosphorylated PDGFRα residues Y₇₆₂ and Y₈₄₉ as well as phosphorylated AKT as downstream substrate. While PDGF-AA alone induced strong activation signals at PDGFRα and AKT, titration of Trimer_{WT} strongly reduced the PDGF-AA induced activity of PDGFRα (FIG. 7E). On the contrary, addition of the PDGFRα binding-deficient Trimer_{Mut} did not reduce the activity of PDGFRα in presence of PDGF-AA (FIG. 7E). Thus, HCMV trimer competed directly with PDGF-AA for the binding to PDGFRα and interfered with PDGFRα signaling, which is an important consideration for the design of an effective and safe trimer-based antiviral strategy.

PDGFRα Activation and Signaling

The fibroblast cell line MRC-5 was used to study receptor phosphorylation and downstream signaling. MRC-5 were grown in RPMI media supplemented with 10% FBS, glutamine and antibiotics. Cells were cultured at 37° C. and 5% CO₂. The cells were seeded in M6 well plates, grown to ˜75% confluency and starved overnight prior to stimulation. The day of the assay, cells were stimulated with PDGF-AA (3.7 nM concentration), CMV trimer, or PDGF-AA:CMV trimer at increasing molar ratios. Stimulations were performed at 37° C. for 10 minutes in serum free media. Following treatment, the cells were washed with cold PBS and lysed (lysis buffer: 50 mM Tris HCl (pH 7.4), 150 mM NaCl, 2 mM EDTA, 1% (v/v) NP40, supplemented with protease (Roche) and phosphatase inhibitors (Sigma)). Samples were diluted in loading buffer (Thermo Fisher Scientific) using denaturing conditions, and analyzed by western blotting using a LI-COR® instrument.

Antibodies and Recombinant Proteins

All primary antibodies used in these Examples were purchased from Cell Signaling Technology®. Secondary antibodies used for detection (IRDYES®) were acquired from LI-COR® Biosciences. All antibodies were used at the dilutions recommended by the manufacturer and incubations were performed overnight (primary antibodies) or 1 h at room temperature (LI-COR® antibodies).

Human PDGF-AA used for cell stimulation was purchased from STEMCELL™ Technologies. All other recombinant proteins were produced in-house.

### CONCLUSION

These Examples present structures of the HCMV trimer that reveal unprecedented insights into the architecture of the trimer complex, binding of broadly neutralizing antibodies, the mechanism of trimer-mediated HCMV receptor interaction, and the consequences on cellular signaling pathways. These results have important consequences for the design of trimer-based vaccines and anti-viral therapeutics.

Importantly, these Examples directly show the possibility that the glycan-free surface of gO may be targeted for the development of novel broadly neutralizing antibodies. Additionally, blocking the trimer interaction to PDGFRα and TGFβR3 would also provide a new strategy for targeting HCMV entry. Notably, the trimer makes extensive contacts across multiple interaction sites with PDGFRα and TGFβR3 and attempts to disrupt binding at single sites completely failed to abolish PDGFRα binding (FIG. 5). Instead, multiple interaction sites in gO were demonstrated and need to be targeted simultaneously to block the interaction of HCMV trimer with PDGFRα (FIG. 5G). Thus, a broadly neutralizing antibody with a sufficiently large interaction footprint on gO, including, for example, a multispecific (e.g., bispecific) antibody, may be used to displace the interaction of both PDGFRα and TGFβR3 receptor proteins. Alternatively, for the development of an anti-viral therapeutic, it is possible to utilize the D1-D3 domains of PDGFRα to block trimer binding to endogenous host receptors.

What is claimed is:

1. A modulator of the interaction between the gO subunit of the human cytomegalovirus (HCMV) gHgLgO trimer and PDGFRα that binds to the glycosylation-free surface of the gO subunit and causes a decrease in the binding of the gO subunit to PDGFRα.

2. The modulator of claim 1, wherein the modulator binds to:
(a) one or more of residues R230, R234, V235, K237, and Y238 of the gO subunit;
(b) one or more of residues N81, L82, M84, M86, F109, F111, T114, Q115, R117, K121, and V123 of the gO subunit; and
(c) one or more of residues R336, Y337, K344, D346, N348, E354, and N358 of the gO subunit.

3. A modulator of the interaction between the gO subunit of the HCMV gHgLgO trimer and PDGFRα that binds to:
(a) one or more of residues R230, R234, V235, K237, and Y238 of the gO subunit;
(b) N81, L82, M84, M86, F109, F111, T114, Q115, R117, K121, and V123 of the gO subunit; and
(c) one or more of residues R336, Y337, K344, D346, N348, E354, and N358 of the gO subunit;
and causes a decrease in the binding of the gO subunit to PDGFRα.

4. The modulator of claim 2 or 3, wherein the modulator binds to all 23 of residues R230, R234, V235, K237, Y238, N81, L82, M84, M86, F109, F111, T114, Q115, R117, K121, V123, R336, Y337, K344, D346, N348, E354, and N358 of the gO subunit.

5. The modulator of any one of claims 1-4, wherein the modulator further binds to one or more of residues R47, Y84, and N85 of the gH subunit of HCMV.

6. The modulator any one of claims 1-5, wherein the modulator is a small molecule, an antibody or antigen-binding fragment thereof, a peptide, a mimic, or an inhibitory nucleic acid.

7. The modulator of claim 6, wherein the inhibitory nucleic acid is an ASO or an siRNA.

8. The modulator of claim 6, wherein the antigen-binding fragment is a bis-Fab, an Fv, a Fab, a Fab′-SH, a F(ab′)₂, a diabody, a linear antibody, an scFv, an scFab, a VH domain, or a VHH domain.

9. The modulator of claim 6, wherein the antibody is a bispecific antibody or a multispecific antibody.

10. The modulator of claim 9, wherein the bispecific antibody or multispecific antibody binds to at least three distinct epitopes of the gO subunit.

11. The modulator of claim 10, wherein the at least three distinct epitopes comprise:
(a) a first epitope comprising one or more of residues R230, R234, V235, K237, and Y238 of the gO subunit;
(b) a second epitope comprising one or more of residues N81, L82, M84, M86, F109, F111, T114, Q115, R117, K121, and V123 of the gO subunit; and
(c) a third epitope comprising one or more of residues R336, Y337, K344, D346, N348, E354, and N358 of the gO subunit.

12. The modulator of claim 6, wherein the modulator is a mimic of PDGFRα.

13. A modulator of the interaction between the gO subunit of the HCMV gHgLgO trimer and PDGFRα that binds to the D1 (SEQ ID NO: 11), D2 (SEQ ID NO: 12), and D3 (SEQ ID NO: 13) domains of PDGFRα and causes a decrease in the binding of the gO subunit to PDGFRα.

14. The modulator of claim 13, wherein the modulator binds to:
(a) one or more of residues N103, Q106, T107, E108, and E109 of PDGFRα;
(b) one or more of residues M133, L137, I139, E141, I147, S145, Y206, and L208 of PDGFRα; and
(c) one or more of residues N240, D244, Q246, T259, E263 and K265 of PDGFRα.

15. A modulator of the interaction between the gO subunit of the HCMV gHgLgO trimer and PDGFRα that binds to:
(a) one or more of residues N103, Q106, T107, E108, and E109 of PDGFRα;
(b) one or more of residues M133, L137, I139, E141, I147, S145, Y206, and L208 of PDGFRα; and
(c) one or more of residues N240, D244, Q246, T259, E263 and K265 of PDGFRα;
and causes a decrease in the binding of the gO subunit to PDGFRα.

16. The modulator of claim 14 or 15, wherein the modulator binds to all 19 of residues N103, Q106, T107, E108, E109, M133, L137, I139, E141, I147, S145, Y206, L208, N240, D244, Q246, T259, E263, and K265 of PDGFRα.

17. The modulator of any one of claims 13-16, wherein the modulator further binds to one or more of residues E52, S78, and L80 of PDGFRα.

18. The modulator any one of claims 13-17, wherein the modulator is a small molecule, an antibody or antigen-binding fragment thereof, a peptide, a mimic, or an inhibitory nucleic acid.

19. The modulator of claim 18, wherein the inhibitory nucleic acid is an ASO or an siRNA.

20. The modulator of claim 18, wherein the antigen-binding fragment is a bis-Fab, an Fv, a Fab, a Fab′-SH, a F(ab′)₂, a diabody, a linear antibody, an scFv, an scFab, a VH domain, or a VHH domain.

21. The modulator of claim 18, wherein the antibody is a bispecific antibody or a multispecific antibody.

22. The modulator of claim 21, wherein the bispecific antibody or multispecific antibody binds to at least three distinct epitopes of PDGFRα.

23. The modulator of claim 22, wherein the at least three distinct epitopes comprise:
(a) a first epitope comprising one or more of residues N103, Q106, T107, E108, and E109 of PDGFRα;
(b) a second epitope comprising one or more of residues M133, L137, I139, E141, I147, S145, Y206, and L208 of PDGFRα; and
(c) a third epitope comprising one or more of residues N240, D244, Q246, T259, E263 and K265 of PDGFRα.

24. The modulator of claim 18, wherein the modulator is a mimic of the gO subunit of the HCMV gHgLgO trimer.

25. The modulator of any one of claims 1-24, wherein the modulator decreases binding of the gO subunit of the HCMV gHgLgO trimer to PDGFRα by at least 50%.

26. The modulator of claim 25, wherein the modulator decreases binding of the gO subunit of HCMV trimer to PDGFRα by at least 90%.

27. The modulator of any one of claims 1-26, wherein the modulator decreases binding of the gO subunit of the HCMV gHgLgO trimer to TGFβR3 by at least 50%.

28. The modulator of any one of claims 25-27, wherein the decrease in binding is measured by surface plasmon resonance, biolayer interferometry, or an enzyme-linked immunosorbent assay (ELISA).

29. The modulator of any one of claims 1-28, wherein the modulator has minimal binding with a region of PDGFRα that triggers downstream signaling.

30. The modulator of any one of claims 1-28, wherein the modulator does not bind to a region of PDGFRα that triggers downstream signaling.

31. The modulator of claim 29 or 30, wherein the region of PDGFRα that triggers downstream signaling is a binding site of PDGF.

32. The modulator of any one of claims 1-31, wherein the modulator causes less than a 20% decrease in signaling by PDGFRα compared to signaling in the absence of the modulator.

33. The modulator of claim 32, wherein the modulator does not cause a decrease in signaling by PDGFRα compared to signaling in the absence of the modulator.

34. The modulator of any one of claims 1-33, wherein the modulator causes a decrease in infection of a cell by HCMV relative to infection in the absence of the modulator.

35. The modulator of claim 34, wherein infection is decreased by at least 40%, as measured in a viral infection assay or a viral entry assay using pseudotyped particles.

36. The modulator of any one of claims 1-35, further comprising a pharmaceutically acceptable carrier.

37. A method for treating an HCMV infection in an individual, the method comprising administering to the individual an effective amount of the modulator of any one of claims 1-36, thereby treating the individual.

38. The method of claim 37, wherein the duration or severity of HCMV infection is decreased by at least 40% relative to an individual who has not been administered the modulator.

39. A method for preventing an HCMV infection in an individual, the method comprising administering to the individual an effective amount of the modulator of any one of claims 1-36, thereby preventing an HCMV infection in the individual.

40. A method of prophylaxis against a secondary HCMV infection in an individual, the method comprising administering to the individual an effective amount of the modulator of any one of claims 1-36, thereby preventing a secondary HCMV infection in the individual

41. The method of claim 40, wherein the secondary infection is an HCMV infection of an uninfected tissue.

42. The method of any one of claims 37-41, wherein the individual is immunocompromised, is pregnant, or is an infant.


SEQUENCE LISTING (13 sequences)

SEQ ID NO: 1 - protein, 464 aa, Human cytomegalovirus
MGRKEDMRSISKLFFIISLTVLLFSIINCKVVRPPGRYWLGTVLSTIGKQKLDKFKLEILKQLEREPYTKYFNMTRQHVKNLTMNMTQFPQYYILAGPIRNDSITYLWFDFYSTQLRKPAKYVYSQYNHTAKTITFRPPSCGTVPSMTCLSEMLNVSKRNDTGEQGCGNFTTFNPMFFNVPRWNTKLYVGPTKVNVDSQTIYFLGLTALLLRYAQRNCTHSFYLVNAMSRNLFRVPKYINGTKLKNTMRKLKRKQAPVKEQLEKKTKKSQSTTTPYFSYTTSTALNVTTNATYRVTTSAKRIPTSTIAYRPDSSFMKSIMATQLRDLATWVYTTLRYRNEPFCKPDRNRTAVSEFMKNTHVLIRNETPYTIYGTLDMSSLYYNETMSVENETASDNNETTPTSPSTRFQKTFIDPLWDYLDSLLFLDKIRNFSLQLPAYGNLTPPEHRRAVNLSTLNSLWWWLQ

SEQ ID NO: 2 - protein, 743 aa, Human cytomegalovirus
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 3 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 4 - protein, 10 aa, synthetic construct
VSIDDDTPML

SEQ ID NO: 5 - protein, 5 aa, synthetic construct
QIADF

SEQ ID NO: 6 - protein, 17 aa, synthetic construct
AKRALWTPDQITDITSL

SEQ ID NO: 7 - protein, 345 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPP

SEQ ID NO: 8 - protein, 346 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPP

SEQ ID NO: 9 - protein, 211 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAE

SEQ ID NO: 10 - protein, 199 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPR

SEQ ID NO: 11 - protein, 93 aa, synthetic construct
QLSLPSILPNENEKVVQLNSSFSLRCFGESEVSWQYPMSEEESSDVEIRNEENNSGLFVTVLEVSSASAAHTGLYTCYYNHTQTEENELEGRH

SEQ ID NO: 12 - protein, 85 aa, synthetic construct
IYIYVPDPDVAFVPLGMTDYLVIVEDDDSAIIPCRTTDPETPVTLHNSEGVVPASYDSRQGFNGTFTVGPYICEATVKGKKFQTI

SEQ ID NO: 13 - protein, 104 aa, synthetic construct
FNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVT